(19) United States
(12) Patent Application Publication
(10) Pub. No.: US 2025/0197886 A1
(43) Pub. Date: Jun. 19, 2025

(54) Lentiviral Manufacturing Platform

(71) Applicant: Brammer Bio, LLC, Carlsbad, CA (US)

(72) Inventor: Jessica Tate, East Concord, NY (US)

(21) Appl. No.: 18/846,526

(22) PCT Filed: Mar. 13, 2023
(86) PCT No.: PCT/US2023/015136

§ 371 Date: Sep. 12, 2024

Related U.S. Application Data
(60) Provisional application No. 63/319,690, filed on Mar. 14, 2022.

(51) Int. Cl. C12N 15/86; B01D 15/36; B01D 15/42; B01D 37/00; B01D 39/18; B01D 61/14; B01D 69/02; B01D 69/08; B01D 71/68

(52) U.S. Cl. CPC C12N 15/86; B01D 15/363; B01D 15/426; B01D 37/00; B01D 39/18; B01D 61/145; B01D 69/02; B01D 69/081; B01D 71/68; B01D 2201/182; B01D 2201/184; B01D 2315/10; B01D 2315/16; B01D 2325/0283; C12N 2740/15043; C12N 2740/15051

(57) ABSTRACT

According to embodiments of the present disclosure, apparatuses, systems, and methods (e.g., an end-to-end manufacturing platform) for viral vectors are provided that include upstream production and downstream purification to the drug substance stage that is not impacted by the therapeutic vector payload.

### RELATED APPLICATIONS

This application claims priority from U.S. provisional patent application No. 63/319,690, filed Mar. 14, 2022, which is hereby incorporated herein by reference in its entirety for all purposes.

### BACKGROUND

The viral vector gene therapy industry lacks a good manufacturing practice (GMP)-friendly, scalable, transient transfection suspension manufacturing platform that performs consistently across multiple therapeutic target payloads for viral vectors and is also capable of achieving the required yield and quality standards of biopharmaceutical companies and regulatory agencies.

### SUMMARY

The present disclosure provides compositions, methods, systems and platforms (e.g., an end-to-end manufacturing platform) for the production and purification of lentiviral vectors.

In certain aspects, provided herein is a composition comprising: (a) a lentiviral vector; (b) proline; (c) MgCl₂; and (d) NaCl. In some embodiments the lentiviral vector is VSV-G lentiviral vector or any pseudotype or recombinant derivative thereof. In some embodiments, the lentiviral vector is GaLV-TR lentiviral vector or any pseudotype or recombinant derivative thereof.

In some embodiments, the composition comprises at least 20 mM proline (e.g., at least about 20, 25, 30, 35, 40, 45, 50, 55, 60, 65, 70, 75, 80, 85, 90, 95, or 100 mM proline). In some embodiments, the composition comprises no more than 110 mM proline (e.g., no more than about 110, 105, 100, 95, 90, 85, 80, 75, 70, 65, 60, 55, 50, 45, 40, 35, 30, or 25 mM proline). In certain embodiments, the composition comprises about 25-100 mM proline (e.g., about 25, 30, 35, 40, 45, 50, 55, 60, 65, 70, 75, 80, 85, 90, 95, or 100 mM proline).

In some embodiments, the composition comprises at least 1 mM MgCl₂ (e.g., at least about 1, 2, 3, 4, 5, 6, 7, 8, 9, or 10 mM MgCl₂). In some embodiments, the composition comprises no more than 11 mM MgCl₂ (e.g., no more than about 11, 10, 9, 8, 7, 6, 5, 4, 3, or 2 mM MgCl₂). In certain embodiments, the composition comprises about 2-10 mM MgCl₂ (e.g., about 2, 3, 4, 5, 6, 7, 8, 9, or 10 mM MgCl₂).

In some embodiments, the composition comprises at least 90 mM NaCl (e.g., at least about 90, 95, 100, 105, 110, 115, 120, 125, 130, 135, 140, 145, or 150 mM NaCl). In some embodiments, the composition comprises no more than 160 mM NaCl (e.g., no more than about 160, 155, 150, 145, 140, 135, 130, 125, 120, 115, 110, 105, or 100 mM NaCl). In certain embodiments, the composition comprises about 100-150 mM NaCl (e.g., about 100, 105, 110, 115, 120, 125, 130, 135, 140, 145, or 150 mM NaCl).

In some embodiments, the composition comprises at least 10 mM sodium phosphate buffer at pH 6-7 (e.g., at least about 10, 15, 20, 25, 30, 35, 40, 45, or 50 mM sodium phosphate buffer at pH 6-7). In some embodiments, the composition comprises no more than 60 mM sodium phosphate buffer at pH 6-7 (e.g., no more than about 60, 55, 50, 45, 40, 35, 30, 25, or 20 mM sodium phosphate buffer at pH 6-7). In certain embodiments, the composition comprises about 20-50 mM sodium phosphate buffer at pH 6-7 (e.g., about 20, 25, 30, 35, 40, 45, or 50 mM sodium phosphate buffer at pH 6-7).

In certain embodiments, the composition further comprises lactose. In some embodiments, the composition comprises at least 1% lactose by weight (e.g., at least about 1%, 2%, 3%, 4%, 5%, 6%, 7%, 8%, 9%, or 10% lactose by weight). In some embodiments, the composition comprises no more than 11% lactose by weight (e.g., no more than about 11%, 10%, 9%, 8%, 7%, 6%, 5%, 4%, 3%, or 2% lactose by weight). In certain embodiments, the composition comprises about 2%-10% lactose by weight (e.g., about 2%, 3%, 4%, 5%, 6%, 7%, 8%, 9%, or 10% lactose by weight).

In certain aspects, provided herein is a method of generating a purified lentiviral vector composition comprising performing anion exchange (AEX) chromatography on a composition provided herein (e.g., a composition set forth above) to generate a purified lentiviral vector composition.

In some aspects, provided herein is a method of generating a purified lentiviral vector composition comprising: (i) performing depth filtration on a composition provided herein (e.g., a composition set forth above) to generate a depth filtered lentiviral vector composition; and (ii) performing anion exchange (AEX) chromatography on the depth filtered lentiviral vector composition generated in step (i) to generate a purified lentiviral vector composition.

In certain embodiments of the methods provided herein, the depth filtration step is performed using a depth filter having a gradient of pore sizes starting from about 8-60 micron pores and ending at about 0.45 micron pores. In some embodiments, the largest pore size in the depth filter is at least 20 micron. In some embodiments, the gradient of pore sizes is a continuous gradient. In certain embodiments, the gradient is a discontinuous gradient. In some embodiments, the depth filtration step is performed using a depth filter made of cellulose or modified Polyethylsulfone (mPES) material.

In some embodiments of the methods provided herein, the AEX chromatography step is performed using bind and elute mode. In certain embodiments, AEX step is performed using an equilibrated, positively charged fibrous membrane column.

In some embodiments, the AEX chromatography step is performed with a buffer containing sodium phosphate, lactose, proline, and sodium chloride. In some embodiments, the buffer comprises about 20-50 mM sodium phosphate, about 2-10% lactose, and/or about 25-100 mM proline. In certain embodiments, the buffer further comprises magnesium chloride. In some embodiments, the magnesium chloride is at a concentration of about 10-12 mM. In some embodiments, the buffer is at a pH of about 6.0-6.5. In certain embodiments, the buffer is a column equilibration and/or post-load wash buffer and the NaCl concentration of the buffer is about 150-250 mM. In some embodiments, the buffer is an elution buffer, and the NaCl concentration is about 800-1000 mM. In some embodiments, the buffer is a stripping buffer, and the NaCl concentration is about 1200-2000 mM. In certain embodiments, the AEX chromatography step includes the use of a column equilibration and/or post-load wash buffer provided herein, an elution buffer provided herein, and/or a stripping buffer provided herein.

In certain embodiments of the methods provided herein, the purified lentiviral vector composition is further processed by ultrafiltration and diafiltration (UFDF). In some embodiments the purified lentiviral vector composition is purified using tangential flow filtration (TFF). In some embodiments, the TFF is performed using a modified polyethersulfone (mPES) hollow-fiber membrane. In certain embodiments, the mPES hollow-fiber membrane has about 1.0 mm inner diameter fibers and a pore size of about 100-500 kDa. In some embodiments, the TFF is performed with a harvest volume at about 20-40 L/m². In some embodiments, the TFF is performed with a flow rate controlled by a permeate flux of about 10-50 liters per meter squared per hour (LMMH). In some embodiments, the TFF is performed with an inlet pressure of no more than 8 psi and a transmembrane pressure of no more than 5 psi.

In certain aspects, provided herein is a method of generating a purified lentiviral vector (e.g., a VSV-G lentiviral vector or any pseudotype or recombinant derivative thereof or a GaLV-TR lentiviral vector or any pseudotype or recombinant derivative thereof) composition, comprising: (A) harvesting a lentiviral vector from a culture of lentiviral vector producing cells to generate a lentiviral vector composition; (B) adding a DNase to the lentiviral vector composition; (C) adding additives to the lentiviral vector composition to generate a stabilized lentiviral vector composition comprising: (a) a lentiviral vector; (b) proline; (c) MgCl₂; and (d) NaCl; and (D) performing anion exchange (AEX) chromatography on the stabilized lentiviral vector composition generated in step (C) to generate a purified lentiviral vector composition. In some embodiments, step (C) is performed prior to step (B).

In certain embodiments, the DNase is benzonase nuclease. In some embodiments, about 40-80 U/ml DNase is added to the lentiviral vector composition during step (B).

In some embodiments, the stabilized lentiviral vector composition comprises at least 20 mM proline (e.g., at least about 20, 25, 30, 35, 40, 45, 50, 55, 60, 65, 70, 75, 80, 85, 90, 95, or 100 mM proline). In some embodiments, the stabilized lentiviral vector composition comprises no more than 110 mM proline (e.g., no more than about 110, 105, 100, 95, 90, 85, 80, 75, 70, 65, 60, 55, 50, 45, 40, 35, 30, or 25 mM proline). In certain embodiments, the stabilized lentiviral vector composition comprises about 25-100 mM proline (e.g., about 25, 30, 35, 40, 45, 50, 55, 60, 65, 70, 75, 80, 85, 90, 95, or 100 mM proline).

In some embodiments, the stabilized lentiviral vector composition comprises at least 1 mM MgCl₂ (e.g., at least about 1, 2, 3, 4, 5, 6, 7, 8, 9, or 10 mM MgCl₂). In some embodiments, the stabilized lentiviral vector composition comprises no more than 11 mM MgCl₂ (e.g., no more than about 11, 10, 9, 8, 7, 6, 5, 4, 3, or 2 mM MgCl₂). In certain embodiments, the stabilized lentiviral vector composition comprises about 2-10 mM MgCl₂ (e.g., about 2, 3, 4, 5, 6, 7, 8, 9, or 10 mM MgCl₂).

In some embodiments, the stabilized lentiviral vector composition comprises at least 90 mM NaCl (e.g., at least about 90, 95, 100, 105, 110, 115, 120, 125, 130, 135, 140, 145, or 150 mM NaCl). In some embodiments, the stabilized lentiviral vector composition comprises no more than 160 mM NaCl (e.g., no more than about 160, 155, 150, 145, 140, 135, 130, 125, 120, 115, 110, 105, or 100 mM NaCl). In certain embodiments, the stabilized lentiviral vector composition comprises about 100-150 mM NaCl (e.g., about 100, 105, 110, 115, 120, 125, 130, 135, 140, 145, or 150 mM NaCl).

In some embodiments, the stabilized lentiviral vector composition comprises at least 10 mM sodium phosphate buffer at pH 6-7 (e.g., at least about 10, 15, 20, 25, 30, 35, 40, 45, or 50 mM sodium phosphate buffer at pH 6-7). In some embodiments, the stabilized lentiviral vector composition comprises no more than 60 mM sodium phosphate buffer at pH 6-7 (e.g., no more than about 60, 55, 50, 45, 40, 35, 30, 25, or 20 mM sodium phosphate buffer at pH 6-7). In certain embodiments, the stabilized lentiviral vector composition comprises about 20-50 mM sodium phosphate buffer at pH 6-7 (e.g., about 20, 25, 30, 35, 40, 45, or 50 mM sodium phosphate buffer at pH 6-7).

In certain embodiments, the stabilized lentiviral vector composition further comprises lactose. In some embodiments, the stabilized lentiviral vector composition comprises at least 1% lactose by weight (e.g., at least about 1%, 2%, 3%, 4%, 5%, 6%, 7%, 8%, 9%, or 10% lactose by weight). In some embodiments, the stabilized lentiviral vector composition comprises no more than 11% lactose by weight (e.g., no more than about 11%, 10%, 9%, 8%, 7%, 6%, 5%, 4%, 3%, or 2% lactose by weight). In certain embodiments, the stabilized lentiviral vector composition comprises about 2%-10% lactose by weight (e.g., about 2%, 3%, 4%, 5%, 6%, 7%, 8%, 9%, or 10% lactose by weight).

In some embodiments, the AEX chromatography step is performed with a buffer containing one or more of sodium phosphate, lactose, proline, sodium chloride, or any combination thereof. In some embodiments, the buffer comprises about 20-50 mM sodium phosphate, about 2-10% lactose, and/or about 25-100 mM proline. In certain embodiments, the buffer further comprises magnesium chloride. In some embodiments, the magnesium chloride is at a concentration of about 10-12 mM. In some embodiments, the buffer is at a pH of about 6.0-6.5. In certain embodiments, the buffer is a column equilibration and/or post-load wash buffer and the NaCl concentration of the buffer is about 250 mM. In some embodiments, the buffer is an elution buffer, and the NaCl concentration is about 800-1000 mM. In some embodiments, the buffer is a stripping buffer, and the NaCl concentration is about 1200-2000 mM. In certain embodiments, the AEX chromatography step includes the use of a column equilibration and/or post-load wash buffer provided herein, an elution buffer provided herein, and/or a stripping buffer provided herein. In certain embodiments, the AEX step is performed using an equilibrated, positively charged fibrous membrane column.

In certain embodiments of the methods provided herein, the purified lentiviral vector composition is further processed by ultrafiltration and diafiltration (UFDF). In some embodiments, the purified lentiviral vector composition is purified using tangential flow filtration (TFF). In some embodiments, the TFF is performed using a modified polyethersulfone (mPES) hollow-fiber membrane. In certain embodiments, the mPES hollow-fiber membrane has about 1.0 mm inner diameter fibers and a pore size of about 100-500 kDa. In some embodiments, the TFF is performed with a harvest volume at about 20-40 L/m². In some embodiments, the TFF is performed with a flow rate controlled by a permeate flux of about 10-50 liters per meter squared per hour (LMH). In some embodiments, the TFF is performed with an inlet pressure of no more than 10 psi and a transmembrane pressure of no more than 6 psi.

In certain embodiments, depth filtration is performed on the stabilized lentiviral vector composition prior to step (D). In some embodiments, the depth filtration step is performed using a depth filter having a gradient of pore sizes starting from about 8-60 micron pores and ending at about 0.45 micron pores. In some embodiments, the largest pore size in the depth filter is at least 20 micron. In some embodiments, the gradient of pore sizes is a continuous gradient. I certain embodiments, the gradient is a discontinuous gradient. In some embodiments, the depth filtration step is performed using a depth filter made of cellulose, with or without diatomaceous earth, or modified Polyethylsulfone (mPES) material.

In some embodiments, the methods provided herein further comprise the step of transfecting cells from a cell culture with one or more lentivirus-encoding plasmids to generate a culture of lentiviral vector producing cells. In some embodiments, the cells are HEK293 cells and/or HEK293T cells. In certain embodiments, the transfection is performed using a cationic lipid-based transfection reagent (e.g., the Thermo Fisher Scientific LV-MAX transfection reagent, lipofectamine 2000). In some embodiments, the cells are transfected 3-8 passages after the cells are thawed. In some embodiments, the method further comprises expanding cells to generate the cell culture used in the transfecting step.

### BRIEF DESCRIPTION OF THE DRAWINGS

FIG. 1 is a process flow chart for an exemplary manufacturing platform for the production and purification viral vectors.

FIG. 2 depicts the elution and strip chromatograms for experiment 2 (only the 0.9M NaCl elution step used).

### DETAILED DESCRIPTION

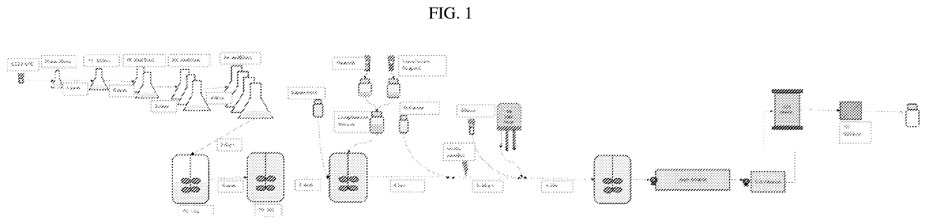

In certain aspects, provided herein are methods and compositions that can be used as part of an end-to-end manufacturing platform, e.g., production and purification, for lentiviral vectors. (See, e.g, FIG. 1 for an exemplary embodiment). In some embodiments, the lentiviral vectors are VSV-G pseudotyped Lentiviral vectors. In some such embodiments, upstream production and downstream purification to the drug substance stage that is not impacted by the therapeutic vector payload. For example, virus production consists of transient transfection of suspension HEK cells using Thermo Fisher Scientific® proprietary cells, media, plasmids, and transfection reagents. Virus purification may also include clarification by depth microfiltration, anion exchange (AEX) chromatography in a bind and elute mode, followed by tangential flow filtration (TFF) to concentrate and formulate the virus.

In some aspects, provided herein are lentiviral vector compositions, such as lentiviral compositions prepared by the methods disclosed herein. Such lentiviral compositions may comprise, in addition to the lentiviral vector, lactose, proline, MgCl2, NaCl, or any combination thereof. In some embodiments, the composition comprises each of the lentiviral vector, lactose, proline, MgCl2, and NaCl. In some embodiments, the lentiviral composition comprises an aqueous sodium phosphate buffer.

In certain aspects, provided herein are methods of generating a purified lentiviral vector composition comprising performing anion exchange chromatography on a lentiviral vector composition disclosed herein. For example, and without limitation, such methods may comprise performing depth filtration on a lentiviral vector composition disclosed herein to generate a depth filtered lentiviral vector composition; and performing anion exchange chromatography on the depth filtered lentiviral vector composition to generate a purified lentiviral vector composition.

In some aspects, provided herein are methods of generating a purified lentiviral vector composition, comprising harvesting a lentiviral vector from a culture of lentiviral vector producing cells to generate a lentiviral vector composition; adding a DNase to the lentiviral vector composition; adding additives to the lentiviral vector composition to generate a stabilized lentiviral vector composition comprising: the lentiviral vector, lactose proline, MgCl₂, and NaCl; and performing anion exchange chromatography on the stabilized lentiviral vector composition. In some embodiments, additives are added to the lentiviral vector composition prior to addition of a DNase.

In certain aspects, provided herein is a composition comprising: (a) a lentiviral vector; (b) proline; (c) MgCl₂; and (d) NaCl. In some embodiments the lentiviral vector is VSV-G lentiviral vector or any pseudotype or recombinant derivative thereof. In some embodiments, the lentiviral vector is GaLV-TR lentiviral vector or any pseudotype or recombinant derivative thereof.

In some embodiments, the composition comprises at least 20 mM proline (e.g., at least about 20, 25, 30, 35, 40, 45, 50, 55, 60, 65, 70, 75, 80, 85, 90, 95, or 100 mM proline). In some embodiments, the composition comprises no more than 110 mM proline (e.g., no more than about 110, 105, 100, 95, 90, 85, 80, 75, 70, 65, 60, 55, 50, 45, 40, 35, 30, or 25 mM proline). In certain embodiments, the composition comprises about 25-100 mM proline (e.g., about 25, 30, 35, 40, 45, 50, 55, 60, 65, 70, 75, 80, 85, 90, 95, or 100 mM proline).

In some embodiments, the composition comprises at least 1 mM MgCl₂ (e.g., at least about 1, 2, 3, 4, 5, 6, 7, 8, 9, or 10 mM MgCl₂). In some embodiments, the composition comprises no more than 11 mM MgCl₂ (e.g., no more than about 11, 10, 9, 8, 7, 6, 5, 4, 3, or 2 mM MgCl₂). In certain embodiments, the composition comprises about 2-10 mM MgCl₂ (e.g., about 2, 3, 4, 5, 6, 7, 8, 9, or 10 mM MgCl₂).

In some embodiments, the composition comprises at least 90 mM NaCl (e.g., at least about 90, 95, 100, 105, 110, 115, 120, 125, 130, 135, 140, 145, or 150 mM NaCl). In some embodiments, the composition comprises no more than 160 mM NaCl (e.g., no more than about 160, 155, 150, 145, 140, 135, 130, 125, 120, 115, 110, 105, or 100 mM NaCl). In certain embodiments, the composition comprises about 100-150 mM NaCl (e.g., about 100, 105, 110, 115, 120, 125, 130, 135, 140, 145, or 150 mM NaCl).

In some embodiments, the composition comprises at least 10 mM sodium phosphate buffer at pH 6-7 (e.g., at least about 10, 15, 20, 25, 30, 35, 40, 45, or 50 mM sodium phosphate buffer at pH 6-7). In some embodiments, the composition comprises no more than 60 mM sodium phosphate buffer at pH 6-7 (e.g., no more than about 60, 55, 50, 45, 40, 35, 30, 25, or 20 mM sodium phosphate buffer at pH 6-7). In certain embodiments, the composition comprises about 20-50 mM sodium phosphate buffer at pH 6-7 (e.g., about 20, 25, 30, 35, 40, 45, or 50 mM sodium phosphate buffer at pH 6-7).

In certain embodiments, the composition further comprises lactose. In some embodiments, the composition comprises at least 1% lactose by weight (e.g., at least about 1%, 2%, 3%, 4%, 5%, 6%, 7%, 8%, 9%, or 10% lactose by weight). In some embodiments, the composition comprises no more than 11% lactose by weight (e.g., no more than about 11%, 10%, 9%, 8%, 7%, 6%, 5%, 4%, 3%, or 2% lactose by weight). In certain embodiments, the composition comprises about 2%-10% lactose by weight (e.g., about 2%, 3%, 4%, 5%, 6%, 7%, 8%, 9%, or 10% lactose by weight).

### Definitions

As used herein, the term “about” refers to a value that is within 10% above or below the value being described. For instance, a value of “about 50 mM” denotes a concentration of from 45 mM to 55 mM.

As used herein, the term “percent by weight per volume” or “% w/v” denotes the percentage weight (in grams) of a single component relative to the total volume of the mixture that contains the component. For instance, 500 mg of a component in a total volume of 8 ml is 6.25% w/v, and 500 mg of a component in a total volume of 5 ml is 10% w/v.

As used herein, the term “viral titer” refers to the number of infectious vector particles, such as “transducing units” (TU), that result in the production of a viral and/or transgene product in a target cell, or “infectious units” (IU), that result in LV particles that enter the cell, reverse transcribe their RNA genome into DNA and integrates into host cell genome. TU=LV particles that enter the cell. Viral titer can be measured by functional assays known in the art.

As used herein, the term “viral vector” refers to a viral particle which has a capability of introducing a nucleic acid molecule into a host. “Lentiviral vectors” includes viral vectors that include sequences derived from HIV-1. A lentiviral vector carrying an exogenous gene(s) may be packaged into an infectious virus particle via virus packaging with the aid of packaging plasmids using specific cell-lines. The infectious virus particle infects a cell to achieve expression of the exogenous gene. A “recombinant” viral vector refers to a viral vector constructed by gene recombinant technologies. A recombinant viral vector can be constructed using methods known in the art, such as by transducing a packaging cell-line with a nucleic acid encoding the viral genome and subsequently isolating newly packaged viral particles. The term “virus” also refers to pseudo-viral particles, i.e., viral particles either without any envelope glycoprotein at their surface, or without a genome and obtained by spontaneous assembling of structural and/or enzymatic proteins of the virus.

Cell culture materials, methods, and techniques are well known to one of skill in the art. For example, a recombinant viral seed stock (e.g., a lentiviral seed stock) is used to infect a confluent host cell population or a host cell population at a certain density (e.g., a 293 cell culture) in a bioreactor at a given multiplicity of infection; the virus is grown in cell culture for a given time and temperature; and the nascent virus progeny harvested in the cell culture fluid. As defined hereinafter, the terms “culture fluid”, “cell culture fluid”, “cell culture media”, “media” and/or “bioreactor fluid” are used interchangeably, and refer to the media or solution in which the cell culture is grown.

As used herein, the term “host cell” or “host cell line” refers to any mammalian cell line which supports replication of a respective virus, such as a wild type, modified or recombinant lentivirus. It will be recognized that any such host cell line is grown in culture to an art known growth phase, followed by infection with a seed stock of the respective virus, then followed by additional culture under physiologically acceptable conditions, resulting in the production of an additional population of virus, which can be harvested by the methods disclosed herein.

### Viral Compositions Containing Additives

Disclosed herein are viral preparations, e.g., lentiviral preparations, exhibiting improved retention properties which prevent the loss of viral vector in subsequent purification steps. Without being bound by any particular theory, and purely for exemplary purposes, the viral preparations disclosed herein may comprise stabilizing additives such as carbohydrates and sugars, amino acids, salts, reducing agents, chelating agents, and/or cationic peptides. Exemplary carbohydrates and sugars include sucrose, trehalose, lactose, mannose, mannitol, glucose, sorbitol, dextrans, polysorbates, polyethylene glycols; such as lactose. Exemplary amino acids include proline, lysine, leucine, histidine, arginine, alanine, isoleucine, methionine, aspartic acid, glutamic acid, and glycine; such as proline. Exemplary salts that may be useful in the stabilized viral preparations disclosed herein include MgCl₂, NaCl, Na₂SO₄, CaCl₂), Na phosphate, Na citrate, Na succinate; e.g., MgCl₂ and/or NaCl. The viral vector preparations may be aqueous mixtures, such as aqueous solutions or suspensions.

In some such embodiments, the amino acid, salt, reducing agent, chelating agent, and/or cationic peptide may be present, e.g., each individually or in any combination, at a concentration of from about 1 mM to about 1 M in the viral preparation (e.g., 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 100 mM, 125 mM, 150 mM, 175 mM, 200 mM, 225 mM, 250 mM, 275 mM, 300 mM, 325 mM, 350 mM, 375 mM, 400 mM, 450 mM, 475 mM, 500 mM, 525 mM, 575 mM, 600 mM, 625 mM, 650 mM, 675 mM, 700 mM, 725 mM, 750 mM, 775 mM, 800 mM, 825 mM, 850 mM, 875 mM, 900 mM, 925 mM, 950 mM, 957 mM, or 1 M). In some embodiments, the concentration is from about 1 mM to about 250 mM, about 2 mM to about 200 mM, or about 10 mM to about 150 mM, (e.g., 2-10 mM, 25-100 mM, or 100-150 mM). In some such embodiments, the carbohydrate/sugar may be present, e.g., at a concentration of 1-10% w/v, such as 1% w/v, 1.5% w/v, 2% w/v, 2.5% w/v, 3% w/v, 3.5% w/v, 4% w/v, 4.5% w/v, 5% w/v, 5.5% w/v, 6% w/v, 6.5% w/v, 7% w/v, 7.5% w/v, 8% w/v, 8.5% w/v, 9% w/v, 9.5% w/v, or 10% w/v.

In some embodiments, the viral composition, e.g., lentiviral composition, comprises 2-10% lactose, 25-100 mM proline, 2-10 mM MgCl2, 100-150 mM NaCl, or any combination thereof. Other buffers useful in conjunction with lentiviral preparations disclosed herein include phosphate buffers, sodium citrate buffers, MES buffers, and MOPS buffers. In some such embodiments, the viral composition, e.g., lentiviral composition, comprises 2-10% w/v lactose, 25-100 mM proline, 2-10 mM MgCl2, 100-150 mM NaCl, in a 20-50 mM Sodium Phosphate buffer at a pH of about 6.0-7.0.

The lentiviral vectors contemplated herein for use with the compositions and methods disclosed herein may include one or more transgenes, e.g., a protein-encoding gene designed for delivery into a host cell and/or integration into the chromosomal DNA thereof. Such lentiviral preparations as are described herein may be used in conjunction with purification techniques, such as filtration and chromatographic procedures, in order to purify lentiviral vectors with improved recovery.

A lentiviral vector may be present within a lentiviral preparation disclosed herein within a range of concentrations. For instance, a lentiviral vector may be present within a lentiviral preparation at a concentration of, e.g., from about 2×10⁸ infectious units per milliliter (IU/mL) to about 1×10⁹ IU/mL (e.g., 2×10⁸ IU/mL, 2.5×10⁸ IU/mL, 3×10⁸ IU/mL, 3.5×10⁸ IU/mL, 4×10⁸ IU/mL, 4.5×10⁸ IU/mL, 5×10′ IU/mL, 5.5×10⁸ IU/mL, 6×10⁸ IU/mL, 6.5×10⁸ IU/mL, 7×10⁸ IU/mL, 7.5×10⁸ IU/mL, 8×10⁸ IU/mL, 8.5×10⁸ IU/mL, 9×10⁸ IU/mL, 9.5×10⁸ IU/mL, or 1×10⁹ IU/mL). When desirable, a lentiviral preparation may contain a lentiviral vector at a concentration of from about 3×10′ IU/mL to about 5×10⁸ IU/mL (e.g., 3×10⁸ IU/mL, 3.5×10⁸ IU/mL, 4×10⁸ IU/mL, 4.5×10⁸ IU/mL, or 5×10⁸ IU/mL).

In certain aspects, provided herein is a method of generating a purified lentiviral vector (e.g., a VSV-G lentiviral vector or any pseudotype or recombinant derivative thereof or a GaLV-TR lentiviral vector or any pseudotype or recombinant derivative thereof) composition, comprising: (A) harvesting a lentiviral vector from a culture of lentiviral vector producing cells to generate a lentiviral vector composition; (B) adding a DNase to the lentiviral vector composition; (C) adding additives to the lentiviral vector composition to generate a stabilized lentiviral vector composition comprising: (a) a lentiviral vector; (b) proline; (c) MgCl₂; and (d) NaCl; and (D) performing anion exchange (AEX) chromatography on the stabilized lentiviral vector composition generated in step (C) to generate a purified lentiviral vector composition. In some embodiments, step (C) is performed prior to step (B).

In certain embodiments, the DNase is benzonase nuclease. In some embodiments, about 40-80 U/ml DNase is added to the lentiviral vector composition during step (B).

In some embodiments, the stabilized lentiviral vector composition comprises at least 20 mM proline (e.g., at least about 20, 25, 30, 35, 40, 45, 50, 55, 60, 65, 70, 75, 80, 85, 90, 95, or 100 mM proline). In some embodiments, the stabilized lentiviral vector composition comprises no more than 110 mM proline (e.g., no more than about 110, 105, 100, 95, 90, 85, 80, 75, 70, 65, 60, 55, 50, 45, 40, 35, 30, or 25 mM proline). In certain embodiments, the stabilized lentiviral vector composition comprises about 25-100 mM proline (e.g., about 25, 30, 35, 40, 45, 50, 55, 60, 65, 70, 75, 80, 85, 90, 95, or 100 mM proline).

In some embodiments, the stabilized lentiviral vector composition comprises at least 1 mM MgCl₂ (e.g., at least about 1, 2, 3, 4, 5, 6, 7, 8, 9, or 10 mM MgCl₂). In some embodiments, the stabilized lentiviral vector composition comprises no more than 11 mM MgCl₂ (e.g., no more than about 11, 10, 9, 8, 7, 6, 5, 4, 3, or 2 mM MgCl₂). In certain embodiments, the stabilized lentiviral vector composition comprises about 2-10 mM MgCl₂ (e.g., about 2, 3, 4, 5, 6, 7, 8, 9, or 10 mM MgCl₂).

In some embodiments, the stabilized lentiviral vector composition comprises at least 90 mM NaCl (e.g., at least about 90, 95, 100, 105, 110, 115, 120, 125, 130, 135, 140, 145, or 150 mM NaCl). In some embodiments, the stabilized lentiviral vector composition comprises no more than 160 mM NaCl (e.g., no more than about 160, 155, 150, 145, 140, 135, 130, 125, 120, 115, 110, 105, or 100 mM NaCl). In certain embodiments, the stabilized lentiviral vector composition comprises about 100-150 mM NaCl (e.g., about 100, 105, 110, 115, 120, 125, 130, 135, 140, 145, or 150 mM NaCl).

In some embodiments, the stabilized lentiviral vector composition comprises at least 10 mM sodium phosphate buffer at pH 6-7 (e.g., at least about 10, 15, 20, 25, 30, 35, 40, 45, or 50 mM sodium phosphate buffer at pH 6-7). In some embodiments, the stabilized lentiviral vector composition comprises no more than 60 mM sodium phosphate buffer at pH 6-7 (e.g., no more than about 60, 55, 50, 45, 40, 35, 30, 25, or 20 mM sodium phosphate buffer at pH 6-7). In certain embodiments, the stabilized lentiviral vector composition comprises about 20-50 mM sodium phosphate buffer at pH 6-7 (e.g., about 20, 25, 30, 35, 40, 45, or 50 mM sodium phosphate buffer at pH 6-7).

In certain embodiments, the stabilized lentiviral vector composition further comprises lactose. In some embodiments, the stabilized lentiviral vector composition comprises at least 1% lactose by weight (e.g., at least about 1%, 2%, 3%, 4%, 5%, 6%, 7%, 8%, 9%, or 10% lactose by weight). In some embodiments, the stabilized lentiviral vector composition comprises no more than 11% lactose by weight (e.g., no more than about 11%, 10%, 9%, 8%, 7%, 6%, 5%, 4%, 3%, or 2% lactose by weight). In certain embodiments, the stabilized lentiviral vector composition comprises about 2%-10% lactose by weight (e.g., about 2%, 3%, 4%, 5%, 6%, 7%, 8%, 9%, or 10% lactose by weight).

In some embodiments, the AEX chromatography step is performed with a buffer containing sodium phosphate, lactose, proline, and sodium chloride. In some embodiments, the buffer comprises about 20-50 mM sodium phosphate, about 2-10% lactose, and/or about 25-100 mM proline. In certain embodiments, the buffer further comprises magnesium chloride. In some embodiments, the magnesium chloride is at a concentration of about 10-12 mM. In some embodiments, the buffer is at a pH of about 6.0-6.5. In certain embodiments, the buffer is a column equilibration and/or post-load wash buffer and the NaCl concentration of the buffer is about 150-250 mM. In some embodiments, the buffer is an elution buffer, and the NaCl concentration is about 800-1000 mM. In some embodiments, the buffer is a stripping buffer, and the NaCl concentration is about 1200-2000 mM. In certain embodiments, the AEX chromatography step includes the use of a column equilibration and/or post-load wash buffer provided herein, an elution buffer provided herein, and/or a stripping buffer provided herein.

In certain embodiments of the methods provided herein, the purified lentiviral vector composition is further processed by ultrafiltration and diafiltration (UFDF). In some embodiments, the purified lentiviral vector composition is purified using tangential flow filtration (TFF). In some embodiments, the TFF is performed using a modified polyethersulfone (mPES) hollow-fiber membrane. In certain embodiments, the mPES hollow-fiber membrane has about 1.0 mm inner diameter fibers and a pore size of about 100-500 kDa. In some embodiments, the TFF is performed with a harvest volume at about 20-40 L/m². In some embodiments, the TFF is performed with a flow rate controlled by a permeate flux of about 10-50 liters per meter squared per hour (LMH). In some embodiments, the TFF is performed with an inlet pressure of no more than 10 psi and a transmembrane pressure of no more than 6 psi.

In certain embodiments, depth filtration is performed on the stabilized lentiviral vector composition prior to step (D). In some embodiments, the depth filtration step is performed using a depth filter having a gradient of pore sizes starting from about 8-60 micron pores and ending at about 0.45 micron pores. In some embodiments, the largest pore size in the depth filter is at least 20 micron. In some embodiments, the gradient of pore sizes is a continuous gradient. I certain embodiments, the gradient is a discontinuous gradient. In some embodiments, the depth filtration step is performed using a depth filter made of cellulose or modified Polyethylsulfone (mPES) material. In certain embodiments, the depth filtration step is performed using an equilibrated, positively charged fibrous membrane column.

In some embodiments, the methods provided herein further comprise the step of transfecting cells from a cell culture with one or more lentivirus-encoding plasmids to generate a culture of lentiviral vector producing cells. In some embodiments, the cells are HEK293 cells and/or HEK293T cells. In certain embodiments, the transfection is performed using cationic lipid-based transfection reagent (e.g., the Thermo Fisher Scientific LV-MAX transfection reagent, lipofectamine 2000). In some embodiments, the cells are transfected 3-8 passages after the cells are thawed. In some embodiments, the method further comprises expanding cells to generate the cell culture used in the transfecting step.

### Viral Vectors

A variety of viral vectors may be used in the methods disclosed herein. For example, and without limitation, the enveloped virus purified according to the methods disclosed herein is an enveloped recombinant virus. In some embodiments, the enveloped virus is a pseudotyped recombinant retrovirus, such as a lentivirus. In some embodiments, the plasmids transfected into the cells of the culture comprise four plasmids, including a plasmid encoding envelope proteins (Env plasmid), which may be derived from virus, e.g., lentivirus, of interest, but may also be derived from other enveloped viruses, such as a plasmid encoding lentiviral Gag and Pol proteins (Gag-Pol plasmid), a plasmid encoding a lentiviral Rev protein (Rev plasmid) and a plasmid comprising a transgene of interest (TOI). In some such embodiments the TOI is encoded by an expression cassette between a lentiviral 3′-LTR and a lentiviral 5′LTR (TOI plasmid). In some embodiments, the envelope protein is derived from the GaLV virus (in particular the modified GaLVTR glycoprotein for lentiviral vectors), from the VSV virus (in particular the VSV-G envelope) or from the measles virus (MV). In certain embodiments, the enveloped virus purified according to the methods disclosed herein is a pseudotyped recombinant retrovirus, more particularly a lentivirus, in which the envelope protein is derived from the GaLV virus (in particular the modified GaLVTR glycoprotein for lentiviral vectors).

As a non-limiting example, one or more of the lenti expression (transfer) plasmid, pLenti6.3/V5-GW/EmGFP, and lenti packaging plasmid, ViraPower™ Lentiviral Packaging Mix, may be used, e.g., packaging plasmids, pLP1, pLP2, and pLP/VSVG with a transgene plasmid of interests at a predetermined appropriate molar ratio. Other lentiviral vectors, including #277.pCCLsin.cPT.hPGK.eGFP,Wpre (277-eGFP) may be used. Other nonlimiting lentiviral plasmids may also be used. Without being bound by theory, both integration competent and integration deficient lentiviral vectors may be used (ICLV and IDLV, respectively). In some such embodiments, a serum-free suspension lentiviral production protocol may be used, wherein such protocols are easy to use, scalable, and highly efficient in delivering lentiviral expression and package vectors into serum-free suspension growing cells at high cell density.

### Cell Culture

The term “cell” as used herein refers includes all types of eukaryotic and prokaryotic cells. In some embodiments, the term refers to eukaryotic cells, especially mammalian cells. In certain exemplary though non-limiting embodiments, the term “cell” is meant to refer to human embryonic kidney (HEK) or human 293 cells, or a variant thereof, such as, a 293 variant that can grow in suspension. In some such embodiments, variants of 293 cells that can grow, proliferate and be transfected in suspension culture, particularly capable of being cultured at high density (e.g., ≥about 2×10⁶ cells/ml, ≥about 3×10⁶ cells/ml, ≥about 4×10⁶ cells/ml, ≥about 6×10⁶ cells/ml, or up to about 20×10⁶ cells/ml).

In some embodiments, the term “high density” when used in the context of culturing cells and conducting transfection workflows, generally refers to a known cell line, or a variant of a known cell line, that can be grown or cultured in an appropriate cell culture medium to densities of ≥about 1×10⁶ cells/ml, ≥about 2×10⁶ cells/ml, ≥about 3×10⁶ cells/ml, or even optionally ≥about 6×10⁶ cells/ml, or ≥about 20×10⁶ cells/ml, while still retaining the ability to be transfected at high efficiency and are able to express a target protein at high.

In some embodiments, the cells are adapted for high density cell culture. This refers to a cell lineage or a (non-clonal) population of cells derived from the same parental cell lineage that has been adapted to grow at high density in a high-density culture medium while retaining cell viability at or above about 80%. Such cells may be isolated or selected out from the parental population of cells by maintaining the cells at high density≥about 40, 50, 60, 70, or 80 sequential passages and gradually replacing the proportion of growth medium with the desired high-density culture medium. Optionally, during the process, different pools of cells may be individually propagated and subjected to the selection procedure while simultaneously assessing transfection efficiency and or lentivirus vector production efficiency, so that non-clonal population of cells may be selected that can be sustained and grown at high density, transfected with high efficiency, and express high levels of a desired recombinant protein. While it will be readily apparent to the skilled practitioner that a variety of cell types and lineages may be subjected to this selection procedure, it has been determined that cell lineages derived from 293 fibroblast cells are particularly amenable to the selection process for being adapted to high density growth conditions. In some scenarios, cells that are adapted to high density growth culture and amenable for use herein will also be capable of being transfected at high efficiency and/or capable of expressing recombinant protein at yield exceeding at least about 200 μg/mL of cell culture up to about 2 mg/mL of cell culture, more typically between about 500 g/ml of cell culture to about 1 mg/mL of cell culture. In some scenarios, cells adapted for high density culture used are capable of being sustained and transfected at densities in the range from about 1×10⁶ to about 20×10⁶ cells/ml, about 2×10⁶ to about 2×10⁶ cells/ml, or about 2.5×10⁶ to about 6×10⁶ cells/ml. In some embodiments, cells may be adapted for high density culture and transfected at densities in the range from about 1×10⁶ to about 20×10⁶, from about 1×10⁶ to about 4×10⁶, from about 1×10⁶ to about 3×10⁶, from about 1×10⁶ to about 2×10⁶.

In some embodiments, the cells are grown in a suspension culture. This includes a cell culture in which the majority or all of the cells in a culture vessel are present in suspension, and the minority or none of the cells in the culture vessel are attached to the vessel surface or to another surface within the vessel. In some embodiments, suspension culture has ≥about 75% of the cells in the culture vessel are in suspension, not attached to a surface on or in the culture vessel. In some embodiments, a suspension culture has ≥about 85% of the cells in the culture vessel are present in suspension, not attached to a surface on or in the culture vessel. In some embodiments, suspension culture has about 95% of the cells in the culture vessel present in suspension, not attached to a surface on or in the culture vessel.

Embodiments disclosed herein include culturing the cells disclosed herein in a number of culture devices such as bioreactors adapted to the culture of cells in suspension. The bioreactor may be a single-use (disposable) or reusable bioreactor. The bioreactor may, for example, be selected from culture vessels or bags and tank reactors. Non-limiting representative bioreactors include a glass bioreactor (e.g. B-DCU® 2 L-10 L, Sartorius), a single-use bioreactor utilizing rocking motion agitation such as wave bioreactor (e.g. Cultibag RM® 10 L-25 L, Sartorius), single use stirrer tank bioreactor (Cultibag STR® 50 L, Sartorius), or stainless steel tank bioreactor. Growth is done under controlled condition (e.g. pH=7.2, p02=50%, 37° C. and a specific agitation according to the system, such as for the culture of HEK 293 cells as disclosed herein).

The methods disclosed herein may also comprise transfection reagents or compositions that facilitate entry of a macromolecule into a cell, as are known in the art. In some such embodiments, the transfection reagent may comprise a cationic lipid. In other embodiments, the transfection reagent may comprise a cationic lipid and one or more neutral/helper lipids as a “lipid aggregate.” Said lipid aggregates may include at least a first cationic lipid and optionally at least a first neutral lipid, wherein said lipid aggregate is suitable for forming a cationic complex with a nucleic acid under aqueous conditions. In some embodiments, the transfection mixture applied to the cultured cells is prepared in parts. For example, and without limitation, the transfection mixture is prepared in two parts. Production plasmids may be diluted into complexation medium. The plasmids may be a mixture of packaging plasmids pLP1, pLP2, and pLP-VSVG, and the transgene plasmid in the appropriate molar ratio, which will be recognized by those of skill in the art. The transfection reagent is then diluted in an appropriate complexion media and added to the plasmid mixture, prior to addition to the cell culture. In some embodiments, duration of incubation of the transfection-plasmid complex is defined as the start of diluted transfection reagent addition to the diluted plasmids to the end of transfection-plasmid mixture addition to cell culture. In some embodiments the mixing process for the transfection-plasmid complex can be performed by means known in the art, such as simple rocking or swirling of a container, in stir tank mixers, and the like prior to addition to culture. Gravity or peristaltic pumps may then be used to transfer transfection-plasmid mixture to cell culture, but the transfer process should be ≤5 mins in duration.

In some embodiments, an enhancer, equilibrated to ambient temperature, may be added to the transfected cell culture. Such enhancers may comprise sodium propionate, sodium butyrate, caffeine, or any combination thereof. In some embodiments, the enhancer optionally comprises valproic acid. In some embodiments, the enhancer does not comprise valproic acid. In some embodiments, valproic acid may be included in the enhancer from about 0.5 to 1 mM. In some embodiments, the enhancer is added at one or more than one time point, such as at the time of transfection (about hour 0) until about 48 hours after transfection. The lentiviral production enhancer may be added at about 4 to 16 hours after transfection to boost cell packaging of lentiviral vectors. In some embodiments, the lentiviral production enhancer may be added at the time of transfection. In some embodiments, the lentiviral production enhancer may be added at the time of transfection and at about 16 hours after transfection. In some embodiments, lentiviral production enhancer may be added from about 4 to 16 hours after transfection. Enhancer can be added to the transfected VPC culture by gravity or peristaltic pump, but the transfer process should be ≤10 mins in duration. In some embodiments, the transfected culture is allowed to incubate until harvest of viral particles, e.g., lentiviral particles.

### Harvest of Viral Particles

The viral particles, e.g., lentiviral particles, are then harvested from the supernatant of the culture (i.e., culture media) according to methods well known in the art. For example, the clarification and purification processes disclosed herein may also comprise one or several steps of treating the sample(s) with one or more nucleases. In some embodiments, the one or more nucleases comprises at least one DNase. In some such embodiments, the DNase is Benzonase. In some embodiments, the nuclease is used in the culture medium (e.g., in the bioreactor) of the producing cells after the plasmid transfection step, in some embodiments, before the clarification and/or purification steps. In some embodiments, the additives disclosed herein are added to the culture medium/harvested supernatant. For exemplary purposes, at harvest additives comprising at least one sugar/carbohydrate, at least one amino acid, at least one salt, or any combination thereof, are added to the culture medium (e.g., in the bioreactor). In some embodiments, the additives comprise 2-10% Lactose, 25-100 mM Proline, 2-10 mM MgCl2 and 100-150 mM NaCl. In certain embodiments, the additives consist of 2-10% lactose, 25-100 mM proline, 2-10 mM MgCl2, and 100-150 mM NaCl, in a 20-50 mM Sodium Phosphate buffer pH 6.0-7.0. The additives may be added before, after, or concurrently with the nuclease.

### Clarification and Purification

Following harvest of the cell culture medium, the subsequent purification process comprises the steps of clarification of the cell culture medium and an anion exchange chromatography.

Clarification may be done by a filtration step, removing cell debris and other impurities from the harvested cell culture medium. Suitable filters may utilize cellulose filters, regenerated cellulose fibers, cellulose fibers combined with inorganic filter aids (e.g., diatomaceous earth, perlite, fumed silica), cellulose filters combined with inorganic filter aids and organic resins, or any combination thereof, and polymeric filters (examples include but are not limited to nylon, polypropylene, polyethersulfone) to achieve effective removal and acceptable recoveries. In some embodiments, a multiple stage process may be used, such as a two or three-stage process comprising a coarse filter(s) to remove large precipitate and cell debris followed by polishing second stage filter(s) with nominal pore sizes greater than 0.2 micron but less than 1 micron. Single stage processes employing, for example, a relatively small pore size filter, or centrifugation may also be used for clarification. More generally, any clarification approach including but not limited to dead-end filtration, microfiltration, centrifugation, or body feed of filter aids (e.g., diatomaceous earth) in combination with dead-end or depth filtration, which provides a filtrate of suitable clarity so as to not foul the membrane and/or resins in the subsequent purification steps may be acceptable for use in the clarification step disclosed herein. In some such embodiments, depth filtration may be used, which employs physical porous filtration mediums that can retain material through the entire depth of the filter. Depth filtration materials and methods are well known to one of skill in the art. For example, the filter material is typically composed of a thick and fibrous cellulosic structure and may further comprise inorganic filter aids such as diatomaceous earth particles embedded in the openings of the fibers. This filter material has a large internal surface area, which is key to particle capture and filter capacity. Such depth filters have a gradient (continuous or discontinuous) of pore sizes starting from 8-60 micron and ending at 0.45 micron, and made of cellulose or modified polyethylsulfone (mPES) material. In some embodiments, the filer is made of cellulose. In some such embodiments, largest pore size is ≥20 micron. Those of skill in the relevant art will recognize that the filter sizing and flow rate required to process the material is dependent on the combination of pore sizes used.

In some embodiments, the filters are flushed beforehand with water, culture medium, or buffer. In some embodiments, the filters are flushed beforehand with anion exchange column (AEX) equilibration buffer. In some embodiments, a post-use flush of the clarification filters is performed. In some such embodiments, the clarified harvest may be collected with this post-flush volume to increase the recovery of virus, e.g., lentivirus, across this step. In some embodiments, at this step an accurate viral titer can be assessed. In certain embodiments, the AEX step is performed using an equilibrated, positively charged fibrous membrane column.

Once the viral preparation has been recovered by clarification as described above, the viral preparation is further purified on an equilibrated, positively charged membrane (AEX; anion exchange column), such as a positively charged fibrous membrane. Membrane materials are well known to one of skill in the art and available from vendors such as Sartorius Corp. (Edgewood, NY), Pall Corp. (East Hills, NY) and Sigma-Aldrich Corp. (St. Louis, MO). Exemplary anion exchange membrane adsorbers include, but are not limited to a Sartobind™ Q membrane adsorber (Sartorius Corp.) and a Mustang™ Q membrane adsorber (Pall Corp.). In general, methods and buffers known from conventional ion exchange chromatography can be directly applied to membrane adsorber chromatography, which are known to one of skill in the art. In certain embodiments, the anion exchange membrane adsorber chromatography is performed at room temperature. Thus, in certain embodiments, the viral preparation is purified via an AEX column, wherein the clarified preparation is loaded onto the AEX column equilibrated with a first pH buffered salt solution (also referred to as a “column equilibration buffer” which may also be used as a post-load wash). The viral preparation is eluted from the AEX column with a second pH buffered salt solution (“the elution buffer”) and the eluted viral fractions are recovered.

In certain embodiments, the first pH buffered salt solution or equilibration buffer is an NaCl or KCL salt solution. The NaCl or KCl is present in solution at an ionic strength between about at least 0.1 M to about 0.4 M. Thus the ionic strengths of the salts include at least 0.1, 0.2, 0.3 and 0.4 M including fractional ionic strengths therebetween. In one particular embodiment, the salt is NaCl and the ionic strength of the NaCl solution is 150 to 250 mM. The buffer solution may be a phosphate buffer, a N-2-Hydroxyethylpiperazine-N′-2-ethanesulfonic acid (HEPES) buffer or a Tris(hydroxymethyl)aminomethane (TRIS) buffer. These buffers in certain embodiments have a pH between about 6.0 to about 8.0, i.e., a pH of at least 6.0, 6.2, 6.4, 6.6, 6.8, 7.0, 7.2, 7.4, 7.6, 7.8, and 8.0 or pH numbers therebetween. In one particular embodiment, the first pH buffered salt solution has a pH of 6.0-6.5. In yet other embodiments, the first buffer of the anion exchange membrane adsorption step has a pKa between 6.0 to 8.5, i.e., a pKa of at least 6.0, 6.2, 6.4, 6.6, 6.8, 7.0, 7.2, 7.4, 7.6, 7.8, 8.0, 8.2, 8.4 and 8.5 or pKa numbers therebetween.

In some embodiments, the equilibration buffer further comprises about 20-50 mM sodium phosphate, or molarity numbers therebetween. In some such embodiments, the equilibration buffer comprises about 2% lactose to about 10% lactose, or percentages therebetween. In some embodiments, the equilibration buffer further comprises about 25-100 mM proline, or molarity numbers therebetween. In some embodiments, the equilibration buffer further comprises about 4-12 mM MgCl₂, or molarity numbers therebetween.

The second pH buffered salt solution (the “elution buffer”) may also comprise the same buffering components as the first (equilibration) buffer. In certain embodiments, the second pH buffered salt solution or equilibration buffer is an NaCl or KCL salt solution. In some embodiments, the salt in the second pH buffered salt solution is NaCl. The NaCl or KCl is present in solution at an ionic strength between about at least 0.5 M to about 1 M. Thus, the ionic strengths of the salts include at least 0.5, 0.6, 0.7, 0.8, 0.9, and 1 M including fractional ionic strengths therebetween. In some such embodiments, the salt is NaCl and the ionic strength of the NaCl solution is 800 to 1000 mM (0.8 to 1.0 M). The buffer solution may be a phosphate buffer, an N-2-Hydroxyethylpiperazine-N′-2-ethanesulfonic acid (HEPES) buffer or a Tris(hydroxymethyl)aminomethane (TRIS) buffer. These buffers in some embodiments have a pH between about 6.0 to about 8.0, i.e., a pH of at least 6.0, 6.2, 6.4, 6.6, 6.8, 7.0, 7.2, 7.4, 7.6, 7.8, and 8.0 or pH numbers therebetween. In some embodiments, the second pH buffered salt solution has a pH of 6.0-6.5. In yet other embodiments, the second buffer of the anion exchange membrane adsorption step has a pKa between 6.0 to 8.5, i.e., a pKa of at least 6.0, 6.2, 6.4, 6.6, 6.8, 7.0, 7.2, 7.4, 7.6, 7.8, 8.0, 8.2, 8.4 and 8.5 or pKa numbers therebetween.

In some embodiments, the elution buffer further comprises about 20-50 mM sodium phosphate, or molarity numbers therebetween. In some such embodiments, the equilibration buffer comprises about 2% lactose to about 10% lactose, or percentages therebetween. In some embodiments, the equilibration buffer further comprises about 25-100 mM proline, or molarity numbers therebetween. In some embodiments, the equilibration buffer further comprises about 4-12 mM MgCl₂, or molarity numbers therebetween.

In some embodiments, the performance of each chromatography step is measured by conductivity and UV absorbance at wavelength 280 nm.

After passing the clarified harvest through the column, the column is washed with equilibration buffer until UV absorbance plateaus near baseline. To elute the viral particles from the AEX column, the salt concentration (ionic strength) of the elution buffer may be increased by linear gradient or in a single step elution process. In some embodiments, the viral particles bound to the AEX column are eluted with elution buffer into a separate container.

Following viral particle elution from the AEX column, the eluted viral particles are diluted in a final buffer composition comprising 20-50 mM Sodium Phosphate, 2-10% lactose, 25-100 mM Proline, 2-10 mM MgCl₂ pH 6.0-6.5, 100-200 mM NaCl. This purified intermediate may be further processed by ultrafiltration and diafiltration (UFDF) using tangential flow filtration (TFF) immediately. In some embodiments, the purified may be stored for up to 24 hours prior to further processing, e.g., at 2-8° C. In some such embodiments, the viral particles are (and/or further) purified via TFF. In general, TFF is a pressure driven process that uses a membrane(s) to separate components in a liquid solution (or suspension), wherein a fluid (the feed flow) is pumped tangentially along the surface of the membrane and an applied pressure serves to force a “portion” of the fluid through the membrane to the filtrate side (of the membrane). In some embodiments, TFF is performed at room temperature. In such a process, the buffer is exchanged and the viral particles are concentrated. In some embodiments, the TFF comprises concentrating the viral particles recovered from the AEX column at least 5 times, followed by at least one buffer exchange. In other embodiments, the TFF comprises concentrating the viral particles recovered from the AEX column at least five to twenty times, followed by at least five, or at least six, buffer exchanges. Still other embodiments involve at least two, at least three, at least four, at least five, or at least six buffer exchanges following the concentration of viral particles recovered from the AEX column.

TFF materials (e.g., hollow fiber, spiral-wound, flat plate) and methods (e.g., ultrafiltration (UF), diafiltration (DF), microfiltration) are well known to one of skill in the art. In some embodiments, the TFF membrane has a 350, 400, 450, 500, 550, 600, 650 or 700 kDa molecular weight cutoff. In some embodiments, the TFF membrane has a pore size 100-500 kD. In some embodiments, the TFF membrane is an mPES hollow fiber membrane.

In some embodiments, the buffer used in the buffer exchange of the TFF is a phosphate buffer, HEPES buffer or TRIS buffer as described above. However, the buffer in certain embodiments may have a concentration of 5 mM to 15 mM, including concentrations of at least 5 mM, 6 mM, 7 mM, 8 mM, 9 mM, 10 mM, 11 mM, 12 mM, 13 mM, 14 mM and 15 mM, and further including mM concentrations therebetween. In certain embodiments, the buffer has a pH of between about 6.0 to 7.5. Thus in one embodiment the buffer has a pH of 6.0, 6.5, 7.0 or 7.5 or fractional pH values therebetween. In some embodiments, the buffer exchange buffer further comprises lactose, proline, and MgCl₂.

In some embodiments, viral particle fractions from the AEX column are pooled, and the pooled solution is concentrated and the buffer exchanged by TFF using a hollow fiber TFF membrane cartridge as described herein. Flow rate across the filter may be controlled by a permeate flux, but held within acceptable inlet pressure (≤10 psi) and transmembrane pressure (≤6 psi). Viral particles may be concentrated up to 20-fold and buffer exchanged into desired buffer using 5-10 diavolumes. In some embodiments, following concentration and buffer exchange, the retentate is collected.

In certain aspects, provided herein is a method of generating a purified lentiviral vector composition comprising performing anion exchange (AEX) chromatography on a composition provided herein (e.g., a composition set forth above) to generate a purified lentiviral vector composition.

In some aspects, provided herein is a method of generating a purified lentiviral vector composition comprising: (i) performing depth filtration on a composition provided herein (e.g., a composition set forth above) to generate a depth filtered lentiviral vector composition; and (ii) performing anion exchange (AEX) chromatography on the depth filtered lentiviral vector composition generated in step (i) to generate a purified lentiviral vector composition.

In certain embodiments of the methods provided herein, the depth filtration step is performed using a depth filter having a gradient of pore sizes starting from about 8-60 micron pores and ending at about 0.45 micron pores. In some embodiments, the largest pore size in the depth filter is at least 20 micron. In some embodiments, the gradient of pore sizes is a continuous gradient. I certain embodiments, the gradient is a discontinuous gradient. In some embodiments, the depth filtration step is performed using a depth filter made of cellulose or modified Polyethylsulfone (mPES) material. In certain embodiments, the depth filtration step is performed using an equilibrated, positively charged fibrous membrane column.

In some embodiments of the methods provided herein, the AEX chromatography step is performed using bind and elute mode.

In some embodiments, the AEX chromatography step is performed with a buffer containing sodium phosphate, lactose, proline, and sodium chloride. In some embodiments, the buffer comprises about 20-50 mM sodium phosphate, about 2-10% lactose, and/or about 25-100 mM proline. In certain embodiments, the buffer further comprises magnesium chloride. In some embodiments, the magnesium chloride is at a concentration of about 10-12 mM. In some embodiments, the buffer is at a pH of about 6.0-6.5. In certain embodiments, the buffer is a column equilibration and/or post-load wash buffer and the NaCl concentration of the buffer is about 150-250 mM. In some embodiments, the buffer is an elution buffer, and the NaCl concentration is about 800-1000 mM. In some embodiments, the buffer is a stripping buffer, and the NaCl concentration is about 1200-2000 mM. In certain embodiments, the AEX chromatography step includes the use of a column equilibration and/or post-load wash buffer provided herein, an elution buffer provided herein, and/or a stripping buffer provided herein.

In certain embodiments of the methods provided herein, the purified lentiviral vector composition is further processed by ultrafiltration and diafiltration (UFDF).

In some embodiments of the methods provided herein the purified lentiviral vector composition (that has been processed by UFDF or that has not been processed by UFDF) is further purified using tangential flow filtration (TFF). In some embodiments, the TFF is performed using a modified polyethersulfone (mPES) hollow-fiber membrane. In certain embodiments, the mPES hollow-fiber membrane has about 1.0 mm inner diameter fibers and a pore size of about 100-500 kDa. In some embodiments, the TFF is performed with an inlet pressure of no more than 8 psi and a transmembrane pressure of no more than 5 psi.

In some embodiments, also provided herein are kits that include an aqueous composition as described herein (e.g., such as an aqueous composition comprising additives as described herein) and optionally a package insert, e.g., a package insert that instructs a user of the kit to express plasmids in the cells of a culture according to a method as described herein. The kits optionally can further include one or more reagents that can be used to make and use (e.g., harvest and/or purify) a viral composition and/or preparation as described herein.

The kits contemplated herein include the use of aqueous compositions as described herein in methods for delivering plasmids encoding viral particles, which may include a transgene, into host cells, and further harvesting and collecting viral compositions disclosed herein. The contemplated viral compositions (e.g., lentiviral compositions) may be used in methods of preventing or treating disease or conditions, and/or delivering transgenes (e.g., genes encoding chimeric antigen receptors, therapeutic peptides or nucleic acids, and the like), and can involve administration of the compositions described herein.

### EXAMPLES

The methods and compositions provided herein will be more readily understood by reference to the following examples, which are included for purposes of illustration of certain aspects and embodiments provided herein, without limitation. The following examples describe an end-to-end manufacturing (production and purification) platform for lentivirus vector using transient transfection of HEK, that can be performed using the platform systems disclosed herein (e.g., according to the exemplary embodiment depicted in FIG. 1).

### Example 1: Expansion of Cells

The production seed train used a vial from one of the VPC banks, grown in Production Medium supplemented with Glutamax. At thaw and at each subsequent passage, culture vessels were inoculated with VPCs at a cell density of 2.5E+05 to 5.5E+05 vc/mL VPCs and grown to cell density of 2E+06 to 6E+06 vc/mL prior to scaling the culture up in the next passage.

### Example 2: Transfection

Transfection of the VPCs occurred at 3 to 8 passages after thaw. Prior to transfection, the culture was supplemented with LV Max® Supplement at a final concentration of 5% v/v. The transfection mixture is prepared in two parts. First, the production plasmids were diluted into 2.5 to 5.0% complexation medium v/v of final VPC culture volume in Viraplex®, OptiMEM®, or similar media can be used as the complexation medium. The total amount of plasmids to use was 2.0 to 2.5 mg/L of final VPC culture volume. The plasmids were a mixture of pLP1, pLP2, pLP-VSVG, and the transgene plasmid in the appropriate molar ratio. The diluted DNA was incubated at ambient temperature (15 to 25° C.) for 2 to at least 60 minutes (mins) as necessary (e.g., 10 to 60 mins). Second, the LV Max transfection reagent was diluted incubated at ambient temperature for ≤15 mins. The diluted transfection reagent was added to the diluted plasmid mixture, mixed briefly, then incubated at ambient temperature. The mixing process for the transfection-plasmid complex can be performed by simple rocking or swirling of container, or on a platform shaker. Gravity or peristaltic pump can be used to transfer transfection-plasmid mixture to cell culture, but the transfer process should be ≤5 mins in duration. Once all the transfection-plasmid mixture was added to the cell culture, it was defined as post-transfection time=0. Enhancer, equilibrated to ambient temperature, was added to the transfected cell culture 4 to 16 hours post transfection (hpt) (e.g., 4 to 6 hpt), to a final concentration of 40 mL/L of final VPC culture volume. Enhancer can be added to the transfected VPC culture by gravity or peristaltic pump. The transfected VPC culture was then allowed to incubate until LV harvest.

### Example 3: Harvesting of Vector

In the bioreactor at harvest, the culture was treated with 40 to 80 U/mL DNase (e.g., Benzonase) and additives are added to the harvest to protect the loss of LV in subsequent purification steps. The additives can be added after or concurrently with the DNase treatment. The additives consisted of 0-10% Lactose, 25-100 mM Proline, 2-10 mM MgCl2 and 100-150 mM NaCl, in a 20-50 mM Sodium Phosphate buffer pH 6.0-7.0. The treated harvest was then clarified through depth filtration. The depth filters had a gradient (continuous or discontinuous) of pore sizes starting from 8-60 micron and ending at 0.45 micron made of cellulose or modified Polyethylsulfone (mPES) material. In some instances, cellulose was the material and the largest pore size was ≥20 micron. The filter sizing and flow rate required to process the material was dependent on the combination of pore sizes used. The filters were flushed beforehand with water, culture medium, or buffer. In some instances, the buffer is AEX Equilibration buffer (described below). This clarified harvest was the first process intermediate at which accurate viral titers were assessed.

### Example 4: Filtration/Purification Step

The clarified harvest was further purified using a positively charged membrane (AEX column) using a bind and elute mode. The AEX column was operated using buffers containing a sodium phosphate (20-50 mM), lactose (2-10%), proline (25-100 mM), and sodium chloride, which concentration was step dependent (see Table 1), at pH 6.0-6.5. Table 1 below provides appropriate salt concentrations for each chromatography step. The performance of each chromatography step was measured by conductivity and UV absorbance at wavelength 280 nm. After passing the clarified harvest through the column, the column was washed with Equilibration buffer until UV absorbance plateaus near baseline. The LV particles bound to the AEX column were eluted with Elution buffer into a separate container.

TABLE 1

| | NaCl | Proline | |
|---|---|---|---|
| | concen. | concen. | Base |
| Step Buffer | (mM) | (mM) | formulation |
| Column | 150 to 250 | 0 | 20-50 mM Sodium |
| Equilibration &/or | | | Phosphate, 2-10% |
| post-load wash | | | Lactose, pH 6.0-6.5 |
| Elution | 800 to 1000 | 50-100 | |
| Strip | 1200 to 2000 | 50-100 | |

The final buffer composition of the LV particles was 20-50 mM Sodium Phosphate, 2-10% lactose, 25-100 mM Proline, 2-10 mM MgCl2 pH 6.0-6.5, 100-200 mM NaCl. At this point the purified LV intermediate may be further processed by ultrafiltration and diafiltration (UFDF) using Tangential Flow Filtration (TFF) immediately.

Lastly, the LV was further purified and formulated via TFF using an mPES hollow-fiber membrane with 1.0 mm inner diameter fibers with a pore size 100-500 kDa, sized-based off harvest volume. Flow rate across the filter was controlled by a permeate flux and pressure. LV was concentrated up to 20-fold and buffer exchanged into desired buffer using 5-10 diavolumes. Following concentrate and buffer exchange, the retentate was collected.

TABLE 2

| | |
|---|---|
| 10x Harvest | 25 mM Sodium Phosphate, 0.6M Proline, 0.06M MgCl2, |
| Additive | pH 6.0 |
| Buffer/Load | |
| adjustment | |
| Buffer | |
| AEX EQ | 50 mM Sodium Phosphate, 200 mM NaCl, 5% Lactose, |
| Buffer | 100 mM Proline, pH 6.0 |
| AEX Elution | 50 mM Sodium Phosphate, 0.9M NaCl, 5% Lactose, 100 |
| Buffer | mM Proline, pH 6.5 |
| AEX Strip | 50 mM Sodium Phosphate, 1.5M NaCl, 5% Lactose, 100 |
| Buffer | mM Proline, pH 6.5 |
| AEX Eluate | 20 mM Sodium Phosphate, 50 mM Proline, 12 mM MgCl2, |
| Dilution | 5% Lactose, pH 6.5 |
| Buffer | |

AEX Membrane tested: As part of building a manufacturing platform for Lentivirus (LV) using triple transfection of suspension HEK293 cells, a capture chromatography was developed. Historically, LV has been purified using anion exchange (AEX) columns in either membrane or monolith formats due to the large and fragile nature of LV and lack of affinity chromatography specific to LV. Past experiences using AEX membrane columns (Mustang Q) resulted in low yields (<20%), or in high column inlet pressures when using AEX monolith column (CIM-Q, 0.5 nm channel) Out of all resins tested, an AEX membrane column conjugated with amines had the highest probability of success. Therefore, LV purification was optimized on an AEX membrane column as described herein.

### Example 5: LV Chromatographic Purification Optimization

Changing variables for each experiment are disclosed herein in their respective experimental and results sections. The procedure described herein was common among all small scale experiments.

Frozen clarified harvests were allowed to equilibrate to ambient temperature and then centrifuged to remove precipitants generated from the freeze/thaw. Proline, lactose, sodium chloride and/or magnesium chloride was added to the clarified harvests at different concentrations, which are listed in each experiment. The AEX column was operated at a flowrate of 5 MV/min in a downward flow, except during the column strip step, which was operated at up-flow for half the buffer volume and downflow for the latter half of the buffer volume. The harvest material that flowed through the column was collected in equal fractions (either 4 or 8). These fractions were sampled separately and then pooled together and sampled again. The pooled fractions were tested to reduce the testing burden. After the harvest material was loaded, the column was washed with Equilibration buffer, and then eluted with various salt strengths. The eluates were collected in containers containing enough Neutralization buffer to reduce NaCl concentrations to ˜200 mM. Following elution, the column was striped with 1.2 or 1.5 M NaCl and the strip effluent was also diluted with Neutralization buffer to reduce the NaCl concentration to ˜200-400 mM. Samples were originally tested for both infectious titer and physical titer, but the infectious titer assay was not performing as expected and so we proceeded with only physical titer testing using the P24 assay to determine LV titers and recovery. All samples were frozen at ≤−65° C. prior to testing.

RESULTS: I (EXPERIMENT 1). The potential of using a fibrous AEX membrane capture column was evaluated. During this evaluation, it was realized that purifications were improved when using Proline instead of Arginine, and Lactose instead of Sorbitol. Therefore, the fibrous AEX membrane evaluation was conducted using proline and/or lactose.

TABLE 3
Harvest Material for EXPERIMENT 1

| | |
|---|---|
| Load | Add equal volume of Adjustment |
| Adjustments | buffer |

TABLE 4
Buffers Used for EXPERIMENT 1

| Buffer | |
|---|---|
| Name | Formulation |
| Load adjust. | 100 mM NaPO4, 5% lactose, 20 mM MgCl2, 20 mM |
| buffer | proline, pH 6.5 |
| EQ/Wash | 50 mM NaPO4, 120 mM NaCl, pH 7.0 |
| buffer | |
| Elution | 20 mM NaPO4, 0.9M NaCl, 5% lactose, 100 mM proline, |
| buffer | pH 6.5 |
| Strip buffer | 20 mM NaPO4, 1.2M NaCl, 5% lactose, 100 mM proline, |
| | pH 6.5; DOM 15 Sep. 2020 |
| Neut./ | 100 mM NaPO4, 5% lactose, 20 mM MgCl2, 20 mM |
| Adjustment | proline, pH 6.5, DOM 15 Sep. 2020 |
| buffer | |

TABLE 5
Volume and Results for EXPERIMENT 1

| | P24 titer | Total | |
|---|---|---|---|
| Sample | (particle/mL) | Particles | Yield |
| AEX Load | 8.64E+08 | 1.71E+11 | N/A |
| AEX FT#1 + 2 | 8.89E+07 | 8.96E+09 | 5% |
| AEX FT#3 + 4 | 1.33E+08 | 1.21E+10 | 7% |
| AEX Wash | LLOQ | LLOQ | ND |
| Eluate#1 (0.4M) | 3.78E+08 | 3.27E+10 | 19% |
| Eluate#2 (0.9M) | 2.34E+08 | 2.36E+10 | 14% |
| AEX Strip | LLOQ | LLOQ | ND |

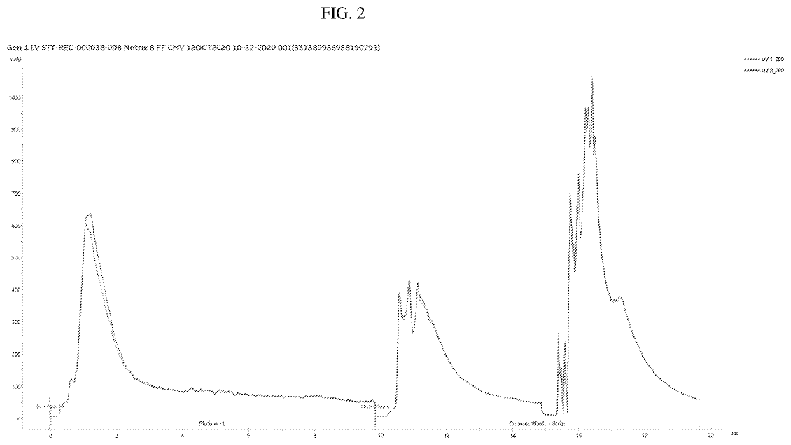

RESULTS: II (Experiment 2). It was determined if more LV particles could be eluted if the 0.4M NaCl elution step was omitted and only the 0.9M NaCl elution step was used. Double the number of fractions of Flowthrough was also collected to determine if the LV in the Flowthrough is the result of LV not binding the column or due to column breakthrough when loading 200 mL of harvest (˜4.58E+11 vp per 1 mL of membrane). The harvest was first treated with Load adjust buffer to final proline concentration of 10 mM by adding equal volume of the Adjustment buffer. Flowthrough was collected in 8×˜13 mL fractions. (See FIG. 2.)

TABLE 6
Harvest Material for Experiment 2

| | |
|---|---|
| Load | Add equal volume of Adjustment |
| Adjustments | buffer |

TABLE 7
Buffers Used for Experiment 2

| Buffer Name | Formulation |
|---|---|
| Load adjust. | 100 mM NaPO4, 5% lactose, 20 mM MgCl2, 20 mM |
| buffer | proline, pH 6.5, |
| EQ/Wash | 50 mM NaPO4, 120 mM NaCl, pH 7.0; DOM 24 Sep. 2020 |
| buffer | |
| Elution | 20 mM NaPO4, 0.9M NaCl, 5% lactose, 100 mM proline, |
| buffer | pH 6.5; |
| Strip buffer | 20 mM NaPO4, 1.2M NaCl, 5% lactose, 100 mM proline, |
| | pH 6.5; |
| Neut buffer | 100 mM NaPO4, 5% lactose, 20 mM MgCl2, 20 mM |
| | proline, pH 6.0, |

TABLE 8
Volume and Results for Experiment 2

| | | | | Res. | Total Res. |
|---|---|---|---|---|---|
| | P24 titer | Total | LV | Host Cell | Host Cell |
| Sample | (particle/mL) | Particles | Recovery | DNA | DNA |
| Load | 4.50E+08 | 9.16E+10 | N/A | 21.1 | 4.30E+03 |
| FT1-4 | LLOQ | LLOQ | 0% | ND | ND |
| FT5 | 5.05E+07 | 1.15E+09 | 1% | ND | ND |
| FT6 | 4.79E+07 | 1.35E+09 | 1% | ND | ND |
| FT7 | 5.79E+07 | 1.64E+09 | 2% | ND | ND |
| FT8 | 7.20E+07 | 2.36E+09 | 3% | ND | ND |
| W | 8.19E+07 | 1.03E+09 | 1% | ND | ND |
| E1 | 8.98E+07 | 1.29E+10 | 14% | 7.25 | 1.04E+03 |
| (0.9M) | | | | | |
| Strip | 2.21E+08 | 3.18E+10 | 35% | 11.4 | 1.64E+03 |

RESULTS: III (Experiment 3). The same conditions as Experiment 2 was used but loaded with less virus (3.66E+11 vp per 1 mL of membrane) to see if that would reduce breakthrough and/or improves recovery of LV in the eluate. Four fractions of equal volume were collected, instead of 8, to keep the fraction volumes the same between II (Experiment 2) and III (STT-Experiment 3).

TABLE 9
Harvest Material for Experiment 3

| | |
|---|---|
| Load | Add equal volume of Adjustment |
| Adjustments | buffer |

TABLE 10
Buffers Used for Experiment 3

| Buffer | |
|---|---|
| Name | Formulation |
| Load adjust. | 100 mM NaPO4, 5% lactose, 20 mM MgCl2, 20 mM |
| buffer | proline, pH 6.5, |
| EQ/Wash | 50 mM NaPO4, 120 mM NaCl, pH 7.0; |
| buffer | |
| Elution | 20 mM NaPO4, 0.9M NaCl, 5% lactose, 100 mM proline, |
| buffer | pH 6.5; |
| Strip buffer | 20 mM NaPO4, 1.2M NaCl, 5% lactose, 100 mM proline, |
| | pH 6.5; |
| Neut buffer | 100 mM NaPO4, 5% lactose, 20 mM MgCl2, 20 mM |
| | proline, pH 6.5, |

TABLE 11
Volume and Results for Experiment 3

| | | | | Res. | Total Res. |
|---|---|---|---|---|---|
| | P24 titer | Total LV | LV vp | Host Cell | Host Cell |
| Sample | (vp/mL) | vp | Recovery | DNA | DNA |
| Load | 7.44E+08 | 7.31E+10 | N/A | 21.4 | 2.10E+03 |
| FT1 + 2 | LLOQ | ND | ND | ND | ND |
| FT3 + 4 | LLOQ | ND | ND | ND | ND |
| W | LLOQ | ND | ND | ND | ND |
| E1 | 8.98E+07 | 1.29E+10 | 18% | 6.63 | 9.51E+02 |
| (0.9M) | | | | | |
| Strip | LLOQ | ND | ND | 18 | 2.59E+03 |

RESULTS: IV (Experiment 4). To improve LV recovery and overall mass balance across purifications step, the concentration of Proline, which is known to reduce aggregation of proteins, was increased to the load to a final concentration of ˜100 mM (previously 10-20 mM), and repeating the step elution with 0.4M, 0.6M. 0.8M and 1.0M NaCl. The salt in the Strip buffer was also increased from 1.2M to 1.5M. The elution buffers were made by mixing the Strip Buffer and Equilibration Buffers in-line on the chromatography skid.

TABLE 12
Harvest Material for Experiment 4

| | |
|---|---|
| Load | Add equal volume of Adjustment |
| Adjustments | buffer |

TABLE 13
Buffers Used for Experiment 4

| Buffer Name | Formulation |
|---|---|
| Load adjust. | 40 mM NaPO4, 8 mM MgCl2, 0.2M proline, pH 6.5, |
| buffer | |
| EQ/Wash | 50 mM NaPO4, pH 7.0; |
| buffer | |
| Strip buffer | 20 mM NaPO4, 1.5M NaCl, 5% lactose, 100 mM |
| | proline, pH 6.5; |
| Neut./ | 100 mM NaPO4, 20 mM MgCl2, 20 mM proline, pH 6.5, |
| Adjustment | |
| buffer | |

TABLE 14
Volume and Results for Experiment 4

| | P24 titer | Total | LV |
|---|---|---|---|
| Sample | (particle/mL) | Particles | Recovery |
| Load | 3.71E+08 | 3.62E+10 | N/A |
| FT1 + 2 | LLOQ | ND | ND |
| FT3 | LLOQ | ND | ND |
| FT4 | 4.83E+07 | 1.42E+09 | 4% |
| Wash | LLOQ | ND | ND |
| E1 | LLOQ | ND | ND |
| (0.4M) | | | |
| E2 | LLOQ | ND | ND |
| (0.6M) | | | |
| E3 | LLOQ | ND | ND |
| (0.8M) | | | |
| E4 | LLOQ | ND | ND |
| (1.0M) | | | |
| Strip | LLOQ | ND | ND |

RESULTS: V (Experiment 5). Repetition of IV (Experiment 4) except only two elutions were performed: 0.4M and 0.9M NaCl. Also, using new Neut. Buffer with lower MgCl₂ and higher proline to ensure that virus is not aggregating and that MgCl₂ is not interfering with the assay.

TABLE 15
Harvest Material for V (Experiment 5)

| | |
|---|---|
| Load | Add equal volume of Adjustment |
| Adjustments | buffer |

TABLE 16
Buffers Used for V (Experiment 5)

| Buffer Name | Formulation |
|---|---|
| Load adjust. | 40 mM NaPO4, 8 mM MgCl2, 0.2M proline, pH 6.5, |
| buffer | |
| EQ/Wash | 50 mM NaPO4, 120 mM NaCl, 5% lactose, pH 6.5; |
| buffer | |
| Elution | 20 mM NaPO4, 0.4M NaCl, 5% lactose, 100 mM proline, |
| buffer #1 | pH 6.5; |
| Elution | 20 mM NaPO4, 0.9M NaCl, 5% lactose, 100 mM proline, |
| buffer #2 | pH 6.5; |
| Strip | 20 mM NaPO4, 1.2M NaCl, 5% lactose, 100 mM proline, |
| buffer | pH 6.5 |
| Neut./ | 10 mM NaPO4, 5% lactose, 12 mM MgCl2, 120 mM |
| Adjustment | proline, pH 6.5, DOM 23 Oct. 2020 |
| buffer | |

TABLE 17
Volume and Results for V (Experiment 5)

| | | | | Res. Host | Total Res. |
|---|---|---|---|---|---|
| | P24 titer | Total | LV | Cell | Host Cell |
| Sample | (particle/mL) | Particles | Recovery | DNA | DNA |
| Load | 8.00E+08 | 8.18E+10 | N/A | 21.1 | 4.30E+03 |
| FT1 + 2 | LLOQ | LLOOQ | ND | ND | ND |
| FT3 + 4 | 1.02E+08 | 5.56E+09 | 7% | ND | ND |
| W | LLOQ | LLOQ | ND | ND | ND |
| E1 | 1.54E+08 | 1.15E+10 | 14% | ND | ND |
| (0.4M) | | | | | |
| E2 | 8.58E+08 | 6.35E+10 | 78% | ND | ND |
| (0.9M) | | | | | |
| Strip | LLOQ | LLOQ | ND | ND | ND |

RESULTS: VI (Experiment 6). Repetition of V (Experiment 5) except lactose concentration was increased in the harvest from 2.5% to 5%.

TABLE 18
Harvest Material for Experiment 5

| | |
|---|---|
| Load | Add equal volume of Adjustment |
| Adjustments | buffer |

TABLE 19
Buffers Used for Experiment 6

| Buffer Name | Formulation |
|---|---|
| Load adjust. | 40 mM NaPO4, 8 mM MgCl2, 0.2M proline, 10% lactose |
| buffer | pH 6.5, |
| EQ/Wash | 50 mM NaPO4, 120 mM NaCl, 5% lactose, pH 6.5; |
| buffer | |
| Elution | 20 mM NaPO4, 0.4M NaCl, 5% lactose, 100 mM proline, |
| buffer #1 | pH 6.5; |
| Elution | 20 mM NaPO4, 0.9M NaCl, 5% lactose, 100 mM proline, |
| buffer #2 | pH 6.5; |
| Strip buffer | 20 mM NaPO4, 1.2M NaCl, 5% lactose, 100 mM proline, |
| | pH 6.5; |
| Neut. buffer | 10 mM NaPO4, 5% lactose, 12 mM MgCl2, 120 mM |
| | proline, pH 6.5, |

TABLE 20
Volume and LV Titer Results for Experiment 6

| | P24 titer | Total | |
|---|---|---|---|
| Sample | (particle/mL) | Particles | LV Recovery |
| Load | 2.99E+08 | 6.08E+10 | N/A |
| FT1 + 2 | LLOQ | ND | ND |
| FT3 + 4 | 2.64E+08 | 1.44E+10 | 24% |
| W | LLOQ | ND | ND |
| E1 | LLOQ | ND | ND |
| (0.4M) | | | |
| E2 | 5.08E+08 | 3.77E+10 | 62% |
| (0.9M) | | | |
| Strip | LLOQ | ND | ND |

RESULTS: VII (Experiment 7). Removing lactose completely from the harvest load was tested for increasing recovery of all LV particles in the 0.9 M elution. By removing lactose, it was possible to make a more concentrated stock solution of load adjustment buffer. Sodium phosphate concentrations were raised in all buffers to increase the pH buffering capacity. At this point, a larger version of this column was tested; 15 mL.

TABLE 21
Harvest Material for Experiment 7

| | |
|---|---|
| Load | Add 200 mL of Adjustment buffer |
| Adjustment | per 1 L of harvest |

TABLE 22
Buffers Used for Experiment 7

| Buffer Name | Formulation |
|---|---|
| Load adjust. | 125 mM PBS, 0.6M Proline, 60 mM MgCl2, pH 6.0 |
| buffer | |
| EQ/Wash | 50 mM NaPO4, 200 mM NaCl, 5% lactose, pH 6.0; |
| buffer | |
| Elution | 50 mM NaPO4, 0.9M NaCl, 5% lactose, 100 mM proline, |
| buffer #1 | pH 6.5; |
| Strip buffer | 50 mM NaPO4, 1.5M NaCl, 5% lactose, 100 mM proline, |
| | pH 6.5; |
| Neut. buffer | 20 mM NaPO4, 5% lactose, 12 mM MgCl2, 50 mM |
| | proline, pH 6.5 |

TABLE 23
Volume and LV Titer Results for Experiment 7

| | P24 titer | Total | LV |
|---|---|---|---|
| Sample | (particle/mL) | Particles | Recovery |
| Load | 7.05E+09 | 2.72E+13 | N/A |
| FT1 | 3.24E+09 | 6.61E+12 | 24% |
| FT2 | 2.83E+09 | 5.15E+12 | 19% |
| Wash | 2.31E+09 | 1.09E+12 | 4% |
| E1 (0.9M) | 1.22E+09 | 3.63E+12 | 13% |
| Strip | 2.61E+08 | 6.62E+11 | 2% |

TABLE 24
Purity Results for Experiment 7

| | | | Total | Residual | Total |
|---|---|---|---|---|---|
| | | Residual | Residual | Host Cell | Residual |
| | | Plasmid | Plasmid | Protein | Host Cell |
| Rec # | Sample | (ng/mL) | (ng) | (ng/mL) | Protein (ng) |
| STT- | Load | 79.1 | 2.99E+08 | 2.26E+05 | N/A |
| REC- | FT1 | 0 | LLOQ | ND | ND |
| 000039- | FT2 | 0 | LLOQ | 2.31E+05 | ND |
| 01 | Wash | 17.9 | LLOQ | ND | ND |
| | E1 | 135 | LLOQ | 3.76E+03 | ND |
| | Strip | 1.04E+05 | 5.08E+08 | 3.44E+02 | ND |

### Example 6: Harvest Treatment and Clarification Optimization

Harvest treatments and clarification to reduce Culture contaminants before chromatography were evaluated. The purpose of these treatments is to remove cells and large debris that may clog chromatography columns and reduce free-floating DNA that would compete with LV on an anion exchange chromatography and lead to DNA impurities in the purified LV product. The typical treatments and clarification used for large-scale LV manufacturing processes involve DNase-treatment, typically utilizing the DNase Benzonase at a concentration of 40-80 U/mL, and clarification through microfiltration using a set of mircrofilters that work in series to first remove large debris (>5 um) and then smaller debris (5 to 0.45 um). A substantial number of manufacturing platforms also include a 3rd clarification step, ultrafiltration in a tangential flow filtration mode after the microfiltration step to improve LV purification on the subsequent column chromatography step. The following characteristics/steps were evaluated and/or optimized:
Efficiency of different DNases to remove DNA impurities,
Clarification through several different microfiltration filter sets
Doing chemical adjustments to the clarified harvest in lieu of adding Ultrafiltration Tangential Flow Filtration as an additional unit operation to improve subsequent chromatography.

Harvests were generated using the following procedure: transfection of Viral Production Cells 1.0 using the LV-Max™ kit components, which includes 3 LV helper plasmids, the lipid-based LV-Max™ transfection reagent, and the LV-Max™ Enhancer, and using a transfer vector containing the LV Self-inactivating Long Terminal Repeats (Sin LTRs), Rev Response Element (RRE), LV psi packaging signal, and GFP as the surrogate therapeutic gene driven by a CMV promoter. Transfection occurred at a cell passage number between 3-8 post cell-vial thaw, either in a shake-flask cell culture container or stirred tank bioreactor, following LV-Max™ kit instructions, and the virus was harvested 48 hrs post transfection. The crude harvest was treated with DNase unless otherwise stated. The DNase-treated harvests were clarified by either centrifugation or by depth filtration/0.45 um filtration. Any other specific experimental conditions that varied between experiments are describe in each subsection below. Samples from these experiments were tested for infectious titer and/or physical titer (P24 ELISA) in most cases, as well as residual extracellular DNA in some experiments. All samples were frozen at ≤−65° C. prior to testing.

RESULTS: DNase-treatment. Experiments were designed to compare potential alternative DNases to Benzonase under normal harvesting conditions, i.e., no pre-treatments of the harvest. All nucleases tested were chosen for their activity level in the normal harvest matrix, with the exception of SAN-HQ, which is optimal at high salt concentrations (>0.4M). The SAN-HQ DNase served a reference point for sub-optimal nuclease activity. The DNase-treated harvests were then cleared of cells by centrifugation following the method described in the Methods section. DNase efficiency was measured by Residual Plasmid and Residual Host Cell DNA qPCR assays as a function of time and/or enzyme concentration. The LLOQ for the Host Cell DNA assay was 1.00E+06 ng/mL.

TABLE 25
Associated LV Production and Harvest records

| Experiment Scale | 50 mL Shake Flasks | 60 mL Shake Flask |
|---|---|---|
| Clarification | Centrifugation | Centrifugation |
| Method | | |

TABLE 26
DNase Experiment #1 Conditions

| Condition | | Nuclease | Incubation |
|---|---|---|---|
| # | Nuclease Type | Concen. (U/mL) | Duration (hr) |
| 1 | Benzonase | 40 | 2 |
| | (Millipore) | | |
| 2 | M-SAN (Articzyme) | 40 | 2 |
| 3 | SAN-HQ (Articzyme) | 40 | 2 |
| 4 | Denerase | 40 | 2 |

TABLE 27
DNase Experiment #1 Results

| | Condition # | | | |
|---|---|---|---|---|
| Replicate | (from Table | P24 titer | IU Titer | Host Cell DNA- |
| # | 3) | (particle/mL) | (IU/mL) | E1A (ng/mL) |
| 1 | 1 | 8.46E+09 | 2.48E+07 | <LLOQ |
| | 2 | 7.70E+09 | 2.09E+07 | <LLOQ |
| | 3 | 9.09E+09 | 2.61E+07 | 1.62E+06 |
| | 4 | 9.93E+09 | 2.55E+07 | <LLOQ |
| 2 | 1 | 1.08E+10 | 3.18E+07 | <LLOQ |
| | 2 | 1.09E+10 | 2.89E+07 | <LLOQ |
| | 3 | 1.26E+10 | 3.20E+07 | 1.74E+06 |
| | 4 | 1.44E+10 | 3.04E+07 | <LLOQ |

TABLE 28
DNase Experiment #2 Conditions

| | | Nuclease Concen. | Incubation |
|---|---|---|---|
| Condition # | Nuclease Type | (U/mL) | Duration (hr) |
| 5 | Benzonase | 25 | 1 |
| 6 | (Millipore) | | 2 |
| 7 | M-SAN (Articzyme) | | 1 |
| 8 | | | 2 |
| 9 | Denerase | | 1 |
| 10 | | | 2 |

TABLE 29
DNase Experiment #2 Results

| Condition # | | | Residual Host | Residual |
|---|---|---|---|---|
| (from Table | P24 titer | IU Titer | Cell DNA - | Plasmid DNA |
| 3) | (particle/mL) | (IU/mL) | E1A (pg/mL) | (pg/mL) |
| 5 | 1.31E+10 | 2.50E+07 | 2.66E+07 | 1.60E+06 |
| 6 | | | 2.82E+07 | 1.52E+06 |
| 7 | 1.24E+10 | 2.46E+07 | 7.53E+07 | 1.51E+06 |
| 8 | | | 5.48E+07 | 1.15E+06 |
| 9 | 1.40E+10 | 2.24E+07 | 1.81E+07 | 1.12E+06 |
| 10 | | | 1.39E+07 | 0.92E+06 |

Most commercially available DNase appear to be relatively interchangeable so long as the harvest composition allows the enzyme the majority of its activity.

Clarification. The optimal filter choice, sizing, flux, and filter flushing parameters for removing cells and other biological contaminants from the DNase-treated harvest while preserving the infectious LV titers was determined. The general consensus across industry is that cellulose filters result in better LV recovery than polypropylene filters. To test this, a Cellulose 8-60 um filter was put in line with either the Cellulose 1-4 um or Polypropylene 5 um filter as the secondary filter (Conditions 1 vs 3 below). The terminal filter in both studies was a Cellulose1.2/0.45. Alternate filters were tested in Conditions 4-6 below.

TABLE 30
Associated LV Production and Harvest

| | | |
|---|---|---|
| Production Scale | 50 L in 50 L SUB (supply run) | 50 L in a 50 L sub |

| | |
|---|---|
| DNase Treatment | 40 U/mL @ 37° C. for 2 hr |

TABLE 31
Clarification Filters Evaluated

| Combin- | Primary | Secondary | Bioburden reduction |
|---|---|---|---|
| ation ID | Depth Filter | Depth Filter | (0.45 um) filter |
| A | Cellulose, 8-60 um | Cellulose, 2-4 um | Cellulose, 1.2/0.5 |
| B | Cellulose, 8-60 um | 5 um | Cellulose, 1.2/0.5 |
| | | Polypropylene | |
| C | Cellulose + DE, | N/A | Cellulose, 1-0.45 |
| | 6-30 um | | um |
| D | N/A | N/A | Cellulose, 1-0.45 |
| | | | um |

TABLE 32
Experiment Conditions

| | | | 1° | 2° | Bioburd. | Pre- | Post- |
|---|---|---|---|---|---|---|---|
| | | Filter | Filter | Filter | Filter | Use | use |
| Condition | Harvest | Combin. | Area | Area | Area | Flush | Flush |
| # | Treatment | ID | (cm2) | (cm2) | (m2) | buffer | buffer |
| 1 | Additives Added | A | 23 | 23 | 25 | WFI | AEX EQ |
| 2 | | | | | | AEX EQ | |
| 3 | | B | 2600 | 1200 | 700 | PBS | |
| 4 | Additive + 0.4M | C | 22 | N/A | 22 | PBS | AEX EQ |
| | NaCl Added | | | | | | |
| 5 | Additives Added | | 22 | N/A | 22 | | |
| 6 | Additive + 0.4M | D | N/A | N/A | 22 | | |
| | NaCl Added | | | | | | |

TABLE 33
Condition #1 & #2 In-Process Measurements

| Experim. | Time | | |
|---|---|---|---|
| Condition | (min) | inlet psi | Flux (LMH) |
| 1 | 0 | 0.2 | N/A |
| | 8 | 5.2 | 170.5 |
| | 9 | 7.4 | 109.1 |
| | 9.5 | 8.5 | 709.1 |
| | 10 | 8.5 | 109.1 |
| | 11 | 9.4 | 109.1 |
| 2 | 0 | 0.2 | N/A |
| | 2 | 0.9 | 250.9 |
| | 8 | 5.2 | 235.9 |
| | 10 | 10.8 | 160.9 |
| | 11 | 13.7 | 248.2 |
| | 12 | 14.4 | 147.3 |
| | 13 | 12.8 | 128.2 |
| | 14 | 14.4 | 62.7 |

TABLE 34
Condition #3 In-Process Measurements

| Experim. | Time | | |
|---|---|---|---|
| Condition | (min) | inlet psi | Flux (LMH) |
| 3 | 0 | 0.1 | |
| | 9.5 | 0.72 | 215.7 |
| | 10.5 | 0.7 | 193.8 |
| | 19 | 1.19 | 186.8 |
| | 31 | 4.3 | 185.7 |
| | 40 | 13.1 | 179.5 |

TABLE 35
Condition #3 In-Process Measurements

| Experim. | Time | | |
|---|---|---|---|
| Condition | (min) | inlet psi | Flux (LMH) |
| 4 | 0 | 0.2 | N/A |
| | 3.5 | 0.2 | 15.4 |
| | 4.5 | 0.7 | 20.2 |
| | 5.5 | 0.7 | 24.7 |
| | 6.5 | 0.8 | 30.0 |
| | 7.5 | 0.9 | 34.7 |
| | 8.5 | 0.7 | 39.4 |
| | 9.5 | 1 | 44.1 |
| | 10.75 | 0.9 | 50.0 |
| | 15.5 | 1.2 | 72.7 |
| | 20.5 | 1.5 | 96.7 |
| | 25.5 | 1.8 | 120.5 |
| | 30.5 | 2 | 144.6 |
| | 35.5 | 2.4 | 169.0 |
| | 40.5 | 2.6 | 194.0 |
| | 45.5 | 2.8 | 216.6 |
| 5 | 0 | 0.3 | N/A |
| | 2.5 | 0.5 | 218.2 |
| | 4.5 | 0.7 | 283.6 |
| | 6.5 | 0.9 | 291.8 |
| | 8.5 | 1 | 291.8 |
| | 10.5 | 1.2 | 287.7 |
| | 15.5 | 1.6 | 284.7 |
| | 20.5 | 2 | 290.7 |
| | 26.5 | 2.5 | 281.8 |
| | 30.5 | 2.8 | 290.5 |
| | 35.5 | 3.6 | 289.1 |
| | 40.5 | 4.4 | 290.2 |
| | 45.5 | 5.2 | 291.8 |

TABLE 36
Clarification Experiment Condition #4 & #5 Results
(No titer for condition #6)

| | IU Titer |
|---|---|
| Condition # | (IU/mL) |
| Pre-filtration | 8.14E+07* |
| 4 (depth) | 5.09E+07 |
| 5 (depth) | 1.51E+07 |
| 4/5 (0.45 um) + | 6.81E+06 |
| flush | |

TABLE 37
Condition #4/5 terminal filter & #6 In-Process Measurements

| Experim. | Time | | |
|---|---|---|---|
| Condition | (min) | inlet psi | Flux (LMH) |
| 6 | 0 | 0.2 | |
| | 3 | 2.6 | 183.6 |
| | 4.5 | 6 | 203.6 |
| | 5.9 | 9.3 | 255.2 |
| | 6.6 | 9.8 | 253.2 |
| | 7.6 | 13 | 182.7 |
| | 9.15 | 11.2 | 95.0 |
| | 11.15 | 10 | 38.2 |
| | 13.15 | 10 | 46.4 |
| | 15.15 | 10 | 38.2 |
| | 17.15 | 10 | 38.2 |
| 4/5 | 0 | 0.2 | |
| (Bio 20 | 2.5 | 1.8 | 162.5 |
| filter) | 4.5 | 1.8 | 189.5 |
| | 8.5 | 1.9 | 186.8 |
| | 10.5 | 2 | 186.8 |
| | 15.5 | 2.1 | 186.5 |
| | 20.5 | 2.4 | 184.9 |
| | 25.5 | 2.6 | 186.0 |
| | 45.6 | 3.1 | 187.4 |
| | 55.75 | 2.9 | 184.6 |

Confirmation Runs. Harvest treatment and clarification parameters at a 50 L Upstream harvest scale were verified. The following conditions were used.

TABLE 38
Harvest Material for Confirmation runs

| Confirm. Run # | 1 | 2 | 3 | 4 |
|---|---|---|---|---|
| Volume (L) | 47.4 | 37.35 | 54.9 | 53.9 |
| Harvest | 40 U DNase, 2 hr | 40 U DNase → 10x | 40 U DNase → 10x | 40 U DNase → 10x |
| Treatment | → 10x Additives | Additives, 2 hr | Additives, 2 hr | Additives, 2 hr |
| Pre-Use Flush | WFI, 20 L | | | |
| Buffer | | | | |
| Post-Use Flush | AEX EQ, ~7-9 L | | | |
| Buffer | | | | |
| Primary Depth | Cellulose 8-60 um | Cellulose 8-60 um | Cellulose 8-60 um | Cellulose 8-60 um |
| Filter | | | | |

| | |
|---|---|
| Secondary Depth | Cellulose filter 2-5 um |
| Filter | |
| 0.45 um Filter | Cellulose filter 0.5-1.2 um |

TABLE 39
Buffers Used for Confirmations Runs

| Buffer Name | Formulation |
|---|---|
| EQ/Wash buffer | 50 mM NaPO4, 200 mM NaCl, 5% lactose, 100 mM |
| | Proline, pH 6.0 |
| 10x Additives | 25 mM NaPO4, 60 mM MgCl2, 0.6M Proline, pH 6.0 |

TABLE 40
Confirmation Runs Filter Load and Flux

| | | Filter Load | | |
|---|---|---|---|---|
| Confirm. | | capacity | Time | Avg Flux |
| Run # | Filter | (L/m2) | (hr) | (LMH) |
| 1 | Primary | 124.2 | 2 | 62.1 |
| | Secondary | 53.2 | | 26.6 |
| | 0.45 | 59.4 | | 29.7 |
| 2 | Primary | 52.7 | 0.5 | 105.5 |
| | Secondary | 37.7 | | 75.3 |
| | 0.45 | 42 | | 84.1 |
| 3 | Primary | 94.5 | 0.5 | 189.1 |
| | Secondary | 67.5 | | 135.1 |
| | 0.45 | 75.4 | | 150.7 |
| 4 | Primary | 83.6 | 0.5 | 167.3 |
| | Secondary | 59.7 | | 119.5 |
| | 0.45 | 66.7 | | 133.3 |

TABLE 41
LV Titers in Clarified Harvests Results for Confirmation Runs

| | | P24 titer | IU Titer |
|---|---|---|---|
| Rec # | Sample | (particle/mL) | (IU/mL) |
| Run 1 | Clarified | 2.75E+09 | 8.9E+06 |
| | Harvest | | |
| Run 2 | Clarified | 4.10E+09 | 1.4E+07 |
| | Harvest | | |
| Run 3 | Clarified | 3.56E+09 | 5.7E+06 |
| | Harvest | | |
| Run 4 | Clarified | 1.19E+09 | 3.8E+06 |
| | Harvest | | |

### Example 7: Harvest Treatment and Clarification Optimization

The manufacturing platform for Lentivirus (LV) using triple transfection of suspension HEK293 cells, involves concentrating and buffer exchange of LV into the final formulation using tangential flow filtration (TFF). There are two filter formats that can be used for TFF, a flat sheet membrane or a hollow fiber (HF) membrane. The HF format is known to require less operating pressure and flow (shear) rates than flat sheet membranes to achieve the same amount of concentration and buffer exchange. Since LV is a relatively fragile virus, HF filters are typically used for LV TFF. Flowrates, pressure, operating pressures, and operating times are all dependent on one another, so the appropriate balance of parameters is required to maximize LV recovery. There are also several pore sizes that the filters can be rated for: 100 kDa, 300 kDa, 500 kDa, or 750 kD. LV should be unable to pass through any of these pore sizes, which is a requirement for successful TFF concentration and buffer exchange. Usually, process design principals indicate that a filter pore size should be picked to be ˜30% smaller than the biologic of interest in order to maximize the number of potential contaminants that can be filtered. However, published studies evaluating lentivirus using different pore-size TFF filters suggest that recovery improves as pore size becomes smaller. Thus, a balance between maximizing LV recovery and maximizing impurity removal is required. The buffer composition of the final formulation buffer can have a significant impact on the performance of TFF and therefore the TFF parameters were tested in both PBS (with no additives), and a formulation buffer that contained cryoprotectants and other stabilizing elements (buffers listed in each experiment below).

For the following experiments the starting material was LV generated by triple transfection of suspension HEK 293 cells, clarified by either depth filtration and purified using an AEX chromatography column. The AEX eluates loaded on the column were concentrated 10- to 20-fold and buffer exchanged using 1 to 10 DV. After buffer exchange the LV-containing TFF retentate was collect. The HF filter was then back flushed with 1.2× hold-up volume of final formulation buffer. The flush and retentate were combined and tested for LV titers. Any other specific experimental conditions that varied between experiments are describe in each subsection below. All samples were frozen at ≤−65° C. prior to testing.

RESULTS: Final TFF.

TABLE 42
TFF Conditions using Hollow Fiber Filter
500 kDa pore size, 1.0 mm i.d. fibers

| | | | Fold | Buffer |
|---|---|---|---|---|
| Condition | | | Concentration | Exchange |
| # | Filter Area | Buffer Used | (by Volume) | Diavolumes |
| 1 | 0.079 m2 | FFB | 20x | 10x |
| 2 | | PBS | 20x | 10x |
| 3 | | FFB | 15x | 5x |
| 4 | | FFB | 10x | 5x |
| 5 | 0.160 cm2 | FFB | 10x | 1x |

TABLE 43
TFF Process measurements

| | | | | | Flux |
|---|---|---|---|---|---|
| | | Inlet | | Processing | (through |
| | Condition | pressure | TMP | Time | filter) |
| Process Step | # | (psi) | (psi) | (min) | (LMH) |
| Concentration | 1 | 10 | 6.5 | 54 | 13.70 |
| | 2 | 6.4 | 5.9 | 53 | 13.85 |
| | 3 | 3.4 | 3.0 | 26 | 27.35 |
| | 4 | 3.7 | 3.1 | 37 | 18.76 |
| | 5 | 3.2 | 3.0 | 212 | 8.95 |
| Buffer | 1 | 3.5 | 3.0 | 27 | 9.95 |
| Exchange | 2 | 3.6 | 3.3 | 23 | 13.01 |
| | 3 | 3.4 | 3.0 | 20 | 14.11 |
| | 4 | 3.6 | 3.0 | ND | ND |
| | 5 | 3.3 | 3.0 | 22 | 8.59 |

TABLE 44
LV Titers of Retentate + Flush

| | | P24 titer | P24 | | IU |
|---|---|---|---|---|---|
| Condition | Process | (particle/ | recovery | IU Titer | recovery |
| # | Step | mL) | % | (IU/mL) | % |
| 1 | TFF Load | 6.16E+09 | N/A | 1.58E+07 | N/A |
| | Ret. + Flush | 7.08E+10 | 93% | 1.55E+08 | 80% |
| 2 | TFF Load | 6.16E+09 | N/A | 1.58E+07 | N/A |
| | Ret. + Flush | LLOQ | ND | LLOQ | ND |
| 3 | TFF Load | 7.15E+09 | N/A | 2.59E+7 | N/A |
| | Ret. + Flush | 8.29E+10 | 100% | 1.96E+08 | 72% |
| 4 | TFF Load | 2.50E+09 | N/A | 7.95E+06 | N/A |
| | Ret. + Flush | 1.80E+10 | 70% | 5.33E+07 | 117% |
| 5 | TFF Load | 1.94E+09 | N/A | 7.20E+06 | N/A |
| | Ret. + Flush | 1.61E+10 | 112% | 4.59E+07 | 81% |

TABLE 45
Residual Levels

| | | Total Res. | |
|---|---|---|---|
| Condition | Total HCP | Plasmid | Total Host |
| # | (ng) | (ng) | Cell DAN |
| 1 | 5.04E+05 | 22.0 | LLOQ |
| 2 | ND | ND | ND |
| 3 | 1.35E+07 | 5320 | 2.70E+10 |
| 4 | 9.73E+05 | 249 | LLOQ |
| 5 | 1.14E+06 | 204 | LLOQ |

What is claimed is:

1. A composition comprising: (a) a lentiviral vector; (b) proline; (c) MgCl₂; and (d) NaCl.

2. The composition of claim 1, comprising 25-100 mM proline.

3. The composition of claim 1 or claim 2, comprising 2-10 mM MgCl₂.

4. The composition of any one of claims 1-3, comprising 100-150 mM NaCl.

5. The composition of any one of claims 1-4, wherein the composition further comprises 20-50 mM sodium phosphate buffer at pH 6-7.

6. The composition of any one of claims 1-5, wherein the lentiviral vector is VSV-G lentiviral vector or any pseudotype or recombinant derivative thereof.

7. The composition of any one of claims 1-5, wherein the lentiviral vector is GaLV-TR lentiviral vector or any pseudotype or recombinant derivative thereof.

8. A method of generating a purified lentiviral vector composition comprising performing anion exchange chromatography on a lentiviral vector composition of any one of claims 1-6 to generate a purified lentiviral vector composition.

9. A method of purifying a lentiviral vector comprising:
(i) performing depth filtration on a lentiviral vector composition of any one of claims 1-6 to generate a depth filtered lentiviral vector composition; and
(ii) performing anion exchange (AEX) chromatography on the depth filtered lentiviral vector composition generated in step (i) to generate a purified lentiviral vector composition.

10. The method of claim 9, wherein step (i) is performed using a depth filter having a gradient of pore sizes starting from about 8-60 micron pores and ending at about 0.45 micron pores.

11. The method of claim 10, wherein the largest pore size in the depth filter is at least 20 micron.

12. The method of claim 10 or 11, wherein the gradient of pore sizes is a continuous gradient.

13. The method of claim 10 or 11, wherein the gradient of pore sizes is a discontinuous gradient.

14. The method of any one of claims 9-13, wherein step (i) is performed using a depth filter made of cellulose or modified Polyethylsulfone (mPES) material.

15. The method of any one of claims 9-14, wherein step (ii) is performed using an equilibrated, positively charged fibrous membrane column.

16. The method of any one of claims 9-15, wherein step (ii) is performed using bind and elute mode.

17. The method of any one of claims 9-18, wherein step (ii) is performed with a buffer containing sodium phosphate, lactose, proline, and sodium chloride.

18. The method of claim 17, wherein the buffer comprises about 20-50 mM sodium phosphate, about 2-10% lactose, and/or about 25-100 mM proline.

19. The method of claim 17 or 18, wherein the buffer further comprises magnesium chloride.

20. The method of claim 19, wherein the magnesium chloride is at a concentration of about 10-12 mM.

21. The method of any one of claims 17-20, wherein the buffer is at a pH of about 6.0-6.5.

22. The method of any one of claims 17-21, wherein the buffer is a column equilibration and/or post-load wash buffer and the NaCl concentration of the buffer is about 150-250 mM.

23. The method of any one of claims 17-21, wherein the buffer is an elution buffer, and the NaCl concentration is about 800-1000 mM.

24. The method of any one of claims 17-21, wherein the buffer is a stripping buffer, and the NaCl concentration is about 1200-2000 mM.

25. The method of any one of claims 9-24, wherein the purified lentiviral vector composition is further processed by ultrafiltration and diafiltration (UFDF).

26. The method of any one of claims 9-25, wherein the purified lentiviral vector composition is further purified using tangential flow filtration (TFF).

27. The method of claim 26, wherein the TFF is performed using a modified polyethersulfone (mPES) hollow-fiber membrane.

28. The method of claim 27 wherein the mPES hollow-fiber membrane has about 1.0 mm inner diameter fibers and a pore size of about 100-500 kDa.

29. The method of claim 28, wherein the TFF is performed with a harvest volume at about 20-40 L/m².

30. The method of any one of claims 26-29, wherein the TFF is performed with a flow rate controlled by a permeate flux of about 10-50 liters per meter squared per hour (LMMH).

31. The method of claim 30, wherein the TFF is performed with an inlet pressure of no more than 8 psi and a transmembrane pressure of no more than 5 psi.

32. A method of generating a purified lentiviral vector composition, comprising:
(A) harvesting a lentiviral vector from a culture of lentiviral vector producing cells to generate a lentiviral vector composition;
(B) adding a DNase to the lentiviral vector composition;
(C) adding additives to the lentiviral vector composition to generate a stabilized lentiviral vector composition comprising: (a) a lentiviral vector; (b) proline; (c) MgCl₂; and (d) NaCl; and
(D) performing anion exchange chromatography on the stabilized lentiviral vector composition generated in step (C) to generate a purified lentiviral vector composition.

33. The composition of claim 32, wherein the lentiviral vector is VSV-G lentiviral vector or any pseudotype or recombinant derivative thereof.

34. The composition of claim 32, wherein the lentiviral vector is GaLV-TR lentiviral vector or any pseudotype or recombinant derivative thereof.

35. The method of any one of claims 32-34, wherein step (C) is performed prior to step (B).

36. The method of any one of claims 32-35, wherein the DNase is benzonase nuclease.

37. The method of any one of claims 32-36, wherein about 40-80 U/ml DNase is added to the lentiviral vector composition during step (B).

38. The method of any one of claims 32-37, wherein the stabilized lentiviral vector composition comprises 25-100 mM proline.

39. The method of any one of claims 32-38, wherein the stabilized lentiviral vector composition comprises 2-10 mM MgCl₂.

40. The method of any one of claims 32-39, wherein the stabilized lentiviral vector composition comprises 100-150 mM NaCl.

41. The method of any one of claims 32-40, wherein the stabilized lentiviral vector composition further comprises 20-50 mM sodium phosphate buffer at pH 6-7.

42. The method of any one of claims 32-41, wherein step (D) is performed using an equilibrated, positively charged fibrous membrane column.

43. The method of any one of claims 32-42, wherein step (D) is performed using bind and elute mode.

44. The method of any one of claims 32-43, wherein step (D) is performed with a buffer containing sodium phosphate, lactose, proline, and sodium chloride.

45. The method of claim 44, wherein the buffer comprises about 20-50 mM sodium phosphate, about 2-10% lactose, and/or about 25-100 mM proline.

46. The method of claim 44 or 45, wherein the buffer further comprises magnesium chloride.

47. The method of claim 46, wherein the magnesium chloride is at a concentration of about 10-12 mM.

48. The method of any one of claims 44-47, wherein the buffer is at a pH of about 6.0-6.5.

49. The method of any one of claims 44-48, wherein the buffer is a column equilibration and/or post-load wash buffer and the NaCl concentration of the buffer is about 150-250 mM.

50. The method of any one of claims 44-48, wherein the buffer is an elution buffer, and the NaCl concentration is about 800-1000 mM.

51. The method of any one of claims 44-48, wherein the buffer is a stripping buffer, and the NaCl concentration is about 1200-2000 mM.

52. The method of any one of claims 32-51, wherein the purified lentiviral vector composition is further processed by ultrafiltration and diafiltration (UFDF).

53. The method of any one of claims 32-52, wherein the purified lentiviral vector composition is further purified using tangential flow filtration (TFF).

54. The method of claim 53, wherein the TFF is performed using a modified polyethersulfone (mPES) hollow-fiber membrane.

55. The method of claim 54 wherein the mPES hollow-fiber membrane has about 1.0 mm inner diameter fibers and a pore size of about 100-500 kDa.

56. The method of claim 55, wherein the TFF is performed with a harvest volume at about 20-40 L/m².

57. The method of any one of claims 53-56, wherein the TFF is performed with a flow rate controlled by a permeate flux of about 10-50 liters per meter squared per hour (LMMH).

58. The method of claim 57, wherein the TFF is performed with an inlet pressure of no more than 8 psi and a transmembrane pressure of no more than 5 psi.

59. The method of any one of claims 32-58, wherein depth filtration is performed on the stabilized lentiviral vector composition prior to step (D).

60. The method of claim 59, wherein the depth filtration is performed using a depth filter having a gradient of pore sizes starting from about 8-60 micron pores and ending at about 0.45 micron pores.

61. The method of claim 60, wherein the largest pore size in the depth filter is at least 20 micron.

62. The method of claim 60 or 61, wherein the gradient of pore sizes is a continuous gradient.

63. The method of claim 60 or 61, wherein the gradient of pore sizes is a discontinuous gradient.

64. The method of any one of claims 59-63, wherein the depth filtration is performed using a depth filter made of cellulose or modified Polyethylsulfone (mPES) material.

65. The method of any one of claims 32-64, further comprising the step of transfecting cells from a cell culture with one or more lentivirus-encoding plasmids to generate the culture of lentiviral vector producing cells prior to step (A).

66. The method of claim 65, wherein the cells are HEK293 cells and/or HEK293T cells.

67. The method of claim 65 or 66 wherein transfection is performed using a cationic lipid-based transfection reagent.

68. The method of any one of claims 65-67, further comprising expanding cells to generate the cell culture used in the transfecting step.
